(19) United States
(12) Patent Application Publication
(10) Pub. No.: US 2014/0037729 A1
(43) Pub. Date: Feb. 6, 2014

(54) COMBINATION OF AN OPIOID AGONIST AND AN OPIOID ANTAGONIST IN THE TREATMENT OF PARKINSON'S DISEASE

(71) Applicants: Michael Hopp, Bad Camberg (DE); Claudia Trenkwalder, Gottingen (DE)

(72) Inventors: Michael Hopp, Bad Camberg (DE); Claudia Trenkwalder, Gottingen (DE)

(73) Assignee: Euro-Celtique, S.A., Luxembourg (LU)

(21) Appl. No.: 13/976,912

(22) PCT Filed: Dec. 27, 2011
(86) PCT No.: PCT/EP2011/074103

§ 371 Date: Oct. 11, 2013

(30) Foreign Application Priority Data
Dec. 28, 2010 (EP) 10197210.7

(51) Int. Cl. A61K 31/485

(52) U.S. Cl. CPC A61K 31/485
USPC 424/468; 514/282

(57) ABSTRACT

The present invention provides a pharmaceutical dosage form comprising an opioid agonist and an opioid antagonist for use in the treatment of Parkinson's disease. The present invention also refers to the use of an opioid agonist and an opioid antagonist in such a dosage form.

### FIELD OF THE INVENTION

The present invention is concerned with a pharmaceutical dosage form comprising an opioid agonist and an opioid antagonist for use in the treatment of Parkinson's disease and/or at least one symptom thereof. The present invention further relates to the use of an opioid agonist in combination with an opioid antagonist in a pharmaceutical dosage form for treating Parkinson's disease and/or at least one symptom thereof.

### BACKGROUND OF THE INVENTION

Parkinson's disease (PD) is a neurodegenerative disease, which is inter alia characterized by hypokinesia, rigor and tremor. Hypokinesia as symptom of PD includes a slowing of physical movement (bradykinesia) and a loss of physical movement (akinesia) in extreme cases. The symptoms are the results of the decreased stimulation of the motor cortex by the basal ganglia, normally caused by the action of dopamine, which is produced in the dopaminergic neurons of the brain (specifically the substantia nigra). PD is both chronic and progressive.

Currently, the treatment of PD is based on counteracting dopaminergic deficiency by using dopaminergics, particularly dopamine agonists or the dopamine precursor L-Dopa (which is also referred to as “levodopa”), or combinations of dopaminergics. Common combinations of dopaminergics used in PD are particularly levodopa and berserazide or levodopa and carbidopa. However, long-term treatment of PD patients with dopaminergics, particularly L-Dopa or dopamine agonists results in dyskinesia. Dyskinesia is a movement disorder which leads to the presence of involuntary movements, similar to tics or chorea of the extremities and/or orofacial and/or axial parts of the body. Dyskinesia observed in L-Dopa treated PD patients is referred to as L-Dopa-induced dyskinesia (LID) and occurs in more than half of PD patients after 5 to 10 years of L-dopa treatment, with the percentage of affected patients increasing over time (for a review see e.g. Encarnacion and Hauser (2008) “Levodopa-induced dyskinesias in Parkinson's disease: etiology, impact on quality of life, and treatments.”; Eur Neurol; 60(2): 57-66).

To date, no effective treatment of LID is available. A report on the use of morphine indicated a decrease in dyskinetic movements at very low doses but, however, an increase in akinesia at higher doses (see Berg et al.; “Reduction of dyskinesia and induction of akinesia induced by morphine in two parkinsonian patients with severe sciatica.”; J Neural Transm 1999; 106(7-8): 725-8). A report on the use of naltrexone indicated that naltrexone in the long term even increases dyskinesias (see Samadi et al.; “Naltrexone in the short-term decreases antiparkinsonian response to L-Dopa and in the long-term increases dyskinesias in drug-naïve parkinsonian monkeys.”; Neuropharmacology 2005; 49(2): 165-73).

Furthermore, PD patients frequently suffer from non-motor symptoms, particularly pain. Pain may be experienced in addition to LIDs or may even be induced by the LIDs. A study by Beiske et al. in 2009 (Beiske A G et al.; “Pain in Parkinson's Disease: Prevalence and characteristics”; Pain 2009 January; 141(1-2): 173-7) showed that 83% of PD patients experienced pain of the following type: musculoskeletal, dystonic, radicular-neuropathic and central neuropathic. Pain can be related to motor fluctuations and the off-period and/or occur independently from fluctuations in PD patients. It seems, however, that pain is rarely treated in such a patient population. In the study referred to above by Beiske et al., only 34% of the patients were reported to be on analgesic medication.

Apart from pain as a non-motor symptom, further non-motor symptoms have been recognised as major factors negatively influencing the quality of life of PD patients. A study by Barone et al. (Barone P et al.; “The PRIAMO study: A multicenter assessment of nonmotor symptoms and their impact on quality of life in Parkinson's disease”; Mov Disord. 2009 Aug. 15; 24(11): 1641-9) showed the prevalence of non-motor symptoms in 98.6% of PD patients. The non-motor symptoms referred to were inter alia pain, a disturbed gastrointestinal system resulting particularly in constipation, a disturbed urogenital system as well as sleep and/or psychiatric problems. Other scales such as the NMSQuest (non-motor symptoms questionnaire) also reliably document non-motor symptoms such as pain, mood and constipation and their influence on the quality of life in PD patients (Chaudhari et al.; Mov Disord. 2010 Apr. 30; 25(6):697-701.

Particularly constipation is nowadays regarded as a major non-motor symptom frequently occurring in PD patients (see e.g. Abbott, and Petrovitch; “Frequency of bowel movements and the future risk of Parkinson's disease.”; Neurology 57(3); 456-62). Constipation is even discussed as a symptom preceding the clinical diagnosis of PD for years (see Jost W (2010), “Gastrointestinal dysfunction in Parkinson's disease.”; J Neurol Sci 289(1-2):69-73 and Savica et al.; “Medical records documentation of constipation preceding Parkinson's disease: a case-control study.”; Neurology 73(21); 1752-8). Clearly, the occurrence of constipation complicates the use of opioids for the treatment of other non-motor symptoms such as pain due to the constipation-inducing effect of opioids.

Thus, there is a strong need in the field to treat PD as well as motor and non-motor symptoms associated with PD, such as particularly pain, whilst at the same time reducing further non-motor symptoms such as e.g. constipation and/or saliva drooling associated with PD. If the patients are on a long-term treatment with L-Dopa, there is furthermore the need for a medication, which decreases the LID, while preferably also reducing further non-motor symptoms such as pain, constipation and/or saliva drooling.

Further, although many patients respond to dopaminergic treatment, said treatment may result in unwanted side effects over time and there is thus the need for an additional therapy resulting in less side effects and/or for a therapy that can replace said dopaminergic treatment.

### OBJECTS AND SUMMARY OF THE INVENTION

Accordingly, it is an object of the present invention to provide a pharmaceutical dosage form comprising an opioid agonist and an opioid antagonist for use in the treatment of Parkinson's disease and/or at least one symptom thereof.

A further object of the present invention relates to the use of an opioid agonist in combination with an opioid antagonist in a pharmaceutical dosage form for the treatment of PD and/or at least one symptom thereof.

These and other objects as they will become apparent from the ensuing description are attained by the subject matter of the independent claims. The dependent claims relate to some of the preferred embodiments of the present invention.

In an especially preferred embodiment, the present invention is concerned with a prolonged release pharmaceutical dosage form comprising an opioid agonist and an opioid antagonist for use in the treatment of Parkinson's disease and/or at least one symptom thereof.

In a preferred embodiment thereof, the opioid agonist is selected from the group comprising morphine, oxycodone, hydromorphone, dihydroetorphine, etorphine, nalbuphine, propoxyphene, nicomorphine, dihydrocodeine, diamorphine, papavereturn, codeine, ethylmorphine, phenylpiperidine, methadone, dextropropoxyphene, buprenorphine, pentazocin, tilidine, tramadol, tapentadol, hydrocodone and pharmaceutically acceptable salts thereof; and the opioid antagonist is selected from the group comprising naltrexone, naloxone, nalmefene, nalorphine, nalbuphine, naloxonazine, methylnaltrexone, ketylcyclazocine, norbinaltorphimine, naltrindole and pharmaceutically acceptable salts thereof.

In a preferred embodiment relating to oral dosage forms according to the present invention, the opioid antagonist is selected from an opioid antagonist that substantially fails to become systemically available if administered orally. Thus, it can be preferred that the opioid antagonist displays an oral bioavailability of less than about 10%, preferably of less than about 5%, more preferably of less than about 3% and most preferably of less than about 2%. Naloxone is particularly preferred in this respect due to a high first pass effect and a very low oral bioavailability, which has been reported to be in the range of equal to or less than 2%.

It can be particularly preferred that the opioid agonist is selected from the group comprising oxycodone, hydromorphone, buprenorphine, dihydroetorphine, nalbuphine and pharmaceutically acceptable salts thereof. It can also be particularly preferred that the opioid antagonist is selected from the group comprising naltrexone, naloxone and nalbuphine and pharmaceutically acceptable salts thereof.

In an especially preferred embodiment, the prolonged release pharmaceutical dosage form comprises as opioid agonist oxycodone or a pharmaceutically acceptable salt thereof and as opioid antagonist naloxone or a pharmaceutically acceptable salt thereof, particularly if the dosage form is an oral dosage form.

In such a preferred embodiment, it can further be preferred that the dosage form comprises oxycodone or a pharmaceutically acceptable salt thereof in an amount range of equivalent to about 1 mg to about 160 mg oxycodone HCl and naloxone or a pharmaceutically acceptable salt thereof in an amount range of equivalent to about 0.5 mg to about 80 mg naloxone HCl.

In said embodiment, the dosage form may comprise oxycodone or a pharmaceutically acceptable salt thereof in an amount of equivalent to about 2.5 mg, to about 5 mg, to about 10 mg, to about 15 mg, to about 20 mg, to about 40 mg, to about 50 mg, to about 60 mg, to about 80 mg, to about 100 mg, to about 120 mg, to about 140 mg, or to about 160 mg oxycodone HCl. Naloxone or a pharmaceutically acceptable salt thereof may be present in an amount of equivalent to about 0.5 mg, to about 1 mg, to about 1.5 mg, to about 2 mg, to about 4 mg, to about 5 mg, to about 10 mg, to about 15 mg, to about 20 mg, to about 40 mg, to about 60 mg, or to about 80 mg naloxone HCl.

It is preferred that a prolonged release dosage form comprising oxycodone and naloxone comprises oxycodone or a pharmaceutically acceptable salt thereof in excess over naloxone or a pharmaceutically acceptable salt thereof (related to the overall amounts of both active agents in the dosage form). It can further be preferred that a dosage form comprising oxycodone and naloxone comprises oxycodone or a pharmaceutically acceptable salt thereof in a ratio ranging from about 25:1 to about 1:1, preferably about 10:1 to about 1:1, more preferably about 5:1 to about 1:1 to naloxone or a pharmaceutically acceptable salt thereof (wherein the absolute amounts of the active agents in the dosage form are referred to). It can also be preferred that a dosage form comprising oxycodone and naloxone comprises oxycodone or a pharmaceutically acceptable salt thereof in a weight ratio of about 25:1, about 10:1, about 5:1, about 4.5:1, about 4:1, about 3.5:1, about 3:1, about 2.5:1, about 2:1, about 1.5:1 or about 1:1 to naloxone or a pharmaceutically acceptable salt thereof.

It is particularly preferred that a dosage form comprising oxycodone and naloxone comprises said oxycodone or said pharmaceutically acceptable salt thereof and said naloxone or said pharmaceutically acceptable salt thereof in about a 2:1 ratio by weight.

Thus, preferred embodiments relate to dosage forms comprising amounts of equivalent to about 2.5 mg oxycodone HCl and about 1.25 mg naloxone HCl; about 5 mg oxycodone HCl and about 2.5 mg naloxone HCl; about 10 mg oxycodone HCl and about 5 mg naloxone HCl; about 20 mg oxycodone HCl and about 10 mg naloxone HCl; about 40 mg oxycodone HCl and about 20 mg naloxone HCl; about 80 mg oxycodone HCl and 40 mg naloxone HCl; and about 160 mg oxycodone HCl and about 80 mg naloxone HCl.

It is preferred that a dosage form comprising oxycodone and naloxone (or pharmaceutical salts thereof) releases in vitro, when measured using the Ph. Eur. Paddle Method at 100 rpm in 0.1 N hydrochloric acid, pH 1.2 at 37° C. and using UV detection at 230 nm, about 5% to about 40% of oxycodone or a pharmaceutically acceptable salt thereof by weight and about 5% to about 40% of naloxone or a pharmaceutically acceptable salt thereof by weight at 15 min; about 20% to about 50% of oxycodone or a pharmaceutically acceptable salt thereof by weight and about 20% to about 50% of naloxone or a pharmaceutically acceptable salt thereof by weight at 1 hour; about 30% to about 60% of oxycodone or a pharmaceutically acceptable salt thereof by weight and about 30% to about 60% of naloxone or a pharmaceutically acceptable salt thereof by weight at 2 hours; about 50% to about 80% of oxycodone or a pharmaceutically acceptable salt thereof by weight and about 50% to about 80% of naloxone or a pharmaceutically acceptable salt thereof by weight at 4 hours; about 70% to about 95% of oxycodone or a pharmaceutically acceptable salt thereof by weight and about 70% to about 95% of naloxone or a pharmaceutically acceptable salt thereof by weight at 7 hours; and more than about 80% of oxycodone or a pharmaceutically acceptable salt thereof by weight and more than about 80% of naloxone or a pharmaceutically acceptable salt thereof by weight at 10 hours.

In a particularly preferred embodiment relating to the in vitro release of a prolonged release dosage form comprising oxycodone and naloxone (or pharmaceutical salts thereof), said dosage form releases in vitro, when measured using the Ph. Eur. Paddle Method at 100 rpm in 0.1 N hydrochloric acid, pH 1.2 at 37° C. and using UV detection at 230 nm, about 10% to about 30% of oxycodone or a pharmaceutically acceptable salt thereof by weight and about 10% to about 30% of naloxone or a pharmaceutically acceptable salt thereof by weight at 15 min; about 30% to about 45% of oxycodone or a pharmaceutically acceptable salt thereof by weight and about 30% to about 45% of naloxone or a pharmaceutically acceptable salt thereof by weight at 1 hour; about 40% to about 60% of oxycodone or a pharmaceutically acceptable salt thereof by weight and about 40% to about 60% of naloxone or a pharmaceutically acceptable salt thereof by weight at 2 hours; about 55% to about 70% of oxycodone or a pharmaceutically acceptable salt thereof by weight and about 55% to about 75% of naloxone or a pharmaceutically acceptable salt thereof by weight at 4 hours; about 75% to about 90% of oxycodone or a pharmaceutically acceptable salt thereof by weight and about 75% to about 90% of naloxone or a pharmaceutically acceptable salt thereof by weight at 7 hours; and more than about 85% of oxycodone or a pharmaceutically acceptable salt thereof by weight and more than about 85% of naloxone or a pharmaceutically acceptable salt thereof by weight at 10 hours.

Further, it can be particularly preferred that a prolonged release dosage form comprising oxycodone and naloxone releases the oxycodone or a pharmaceutically acceptable salt thereof and the naloxone or a pharmaceutically acceptable salt thereof at substantially equal release rates.

In another preferred embodiment, the dosage form comprises as the opioid agonist hydromorphone or a pharmaceutically acceptable salt thereof and as the opioid antagonist naloxone or a pharmaceutically acceptable salt thereof.

In such a preferred embodiment, it can be preferred that the dosage form comprises hydromorphone or a pharmaceutically acceptable salt thereof in an amount of equivalent to about 1 mg to about 64 mg hydromorphone HCl and naloxone or a pharmaceutically acceptable salt thereof in an amount range of equivalent to about 0.5 mg to about 256 mg naloxone HCl. Thus, the dosage form may comprise hydromorphone or a pharmaceutically acceptable salt thereof in an amount of equivalent to about 1 mg, to about 2.5 mg, to about 5 mg, to about 10 mg, to about 20 mg, to about 30 mg, to about 40 mg, to about 50 mg, to about 60 mg, or to about 64 mg hydromorphone HCl. In combination therewith, such a dosage form may comprise naloxone or a pharmaceutically acceptable salt thereof in an amount of equivalent to about 0.5 mg, to about 1 mg, to about 1.5 mg, to about 10 mg, to about 20 mg, to about 40 mg, to about 50 mg, to about 60 mg, to about 80 mg, to about 90 mg, to about 100 mg, to about 120 mg, to about 150 mg, to about 160 mg, to about 180 mg, to about 200 mg, to about 220 mg, to about 240 mg, to about 250 mg or to about 264 mg naloxone HCl.

In preferred embodiments relating to a dosage form comprising hydromorphone and naloxone, the dosage form comprises hydromorphone or a pharmaceutically acceptable salt thereof and naloxone or a pharmaceutically acceptable salt thereof in a 2:1, 1:1, 1:2 or 1:3 ratio by weight. However, the dosage form may also comprise said two active agents (hydromorphone:naloxone) in a 3:1, 4:1, 1:4, or 1:5 ratio by weight.

It is preferred that a dosage form comprising hydromorphone and naloxone (or pharmaceutical salts thereof) releases in vitro, when measured using the Ph. Eur. Paddle Method at 100 rpm in 0.1 N hydrochloric acid, pH 1.2 at 37° C. and using UV detection at 230 nm, about 25% to about 55% of hydromorphone or a pharmaceutically acceptable salt thereof by weight and about 25% to about 55% of naloxone or a pharmaceutically acceptable salt thereof by weight at 1 hour; about 45% to about 75% of hydromorphone or a pharmaceutically acceptable salt thereof by weight and about 45% to about 75% of naloxone or a pharmaceutically acceptable salt thereof by weight at 2 hours; about 55% to about 85% of hydromorphone or a pharmaceutically acceptable salt thereof by weight and about 55% to about 85% of naloxone or a pharmaceutically acceptable salt thereof by weight at 3 hours; about 60% to about 90% of hydromorphone or a pharmaceutically acceptable salt thereof by weight and about 60% to about 90% of naloxone or a pharmaceutically acceptable salt thereof by weight at 4 hours; about 70% to about 100% of hydromorphone or a pharmaceutically acceptable salt thereof by weight and about 70% to about 100% of naloxone or a pharmaceutically acceptable salt thereof by weight at 6 hours; more than about 85% of hydromorphone or a pharmaceutically acceptable salt thereof by weight and more than about 85% of naloxone or a pharmaceutically acceptable salt thereof by weight at 8 hours; and more than about 90% of hydromorphone or a pharmaceutically acceptable salt thereof by weight and more than about 90% of naloxone or a pharmaceutically acceptable salt thereof by weight at 10 hours.

It can particularly be preferred that a dosage form comprising hydromorphone and naloxone (or pharmaceutical salts thereof) releases in vitro, when measured using the Ph. Eur. Paddle Method at 100 rpm in 0.1N hydrochloric acid, pH 1.2 at 37° C. and using UV detection at 230 nm, about 30% to about 50% of hydromorphone or a pharmaceutically acceptable salt thereof by weight and about 30% to about 50% of naloxone or a pharmaceutically acceptable salt thereof by weight at 1 hour; about 50% to about 70% of hydromorphone or a pharmaceutically acceptable salt thereof by weight and about 50% to about 70% of naloxone or a pharmaceutically acceptable salt thereof by weight at 2 hours; about 60% to about 80% of hydromorphone or a pharmaceutically acceptable salt thereof by weight and about 60% to about 80% of naloxone or a pharmaceutically acceptable salt thereof by weight at 3 hours; about 65% to about 85% of hydromorphone or a pharmaceutically acceptable salt thereof by weight and about 65% to about 85% of naloxone or a pharmaceutically acceptable salt thereof by weight at 4 hours; about 75% to about 95% of hydromorphone or a pharmaceutically acceptable salt thereof by weight and about 75% to about 95% of naloxone or a pharmaceutically acceptable salt thereof by weight at 6 hours; more than about 90% of hydromorphone or a pharmaceutically acceptable salt thereof by weight and more than about 90% of naloxone or a pharmaceutically acceptable salt thereof by weight at 8 hours; and more than about 95% of hydromorphone or a pharmaceutically acceptable salt thereof by weight and more than about 95% of naloxone or a pharmaceutically acceptable salt thereof by weight at 10 hours.

In another preferred embodiment, the dosage form comprises as the opioid agonist buprenorphine or a pharmaceutically acceptable salt thereof and/or dihydroetorphine or a pharmaceutically acceptable salt thereof, particularly if the dosage form is a transdermal dosage form.

In another preferred embodiment, the pharmaceutically acceptable salt of the opioid agonist and/or the opioid antagonist is selected from the group comprising the hydrochloride, sulphate, bisulphate, tartrate, nitrate, citrate, bitartrate, phosphate, malate, maleate, hydrobromide, hydroiodide, fumerate and succinate salt. It can be particularly preferred that the salt is the hydrochloride salt.

In another especially preferred embodiment, the present invention relates to an immediate release pharmaceutical dosage form comprising an opioid agonist and an opioid antagonist for use in the treatment of Parkinson's disease and/or at least one symptom thereof.

In other preferred embodiments, the opioid agonist and the opioid antagonist are thus present in an immediate release pharmaceutical dosage form for the treatment of Parkinson's disease and/or at least one symptom thereof with specific active agents (i.e. the lists of opioid agonists and antagonists as described above in certain embodiments), combinations of said two active agents (i.e. the combinations of oxycodone and naloxone; or hydromorphone and naloxone as described above in certain embodiments), corresponding amounts (i.e. the amounts of oxycodone and/or naloxone and/or hydromorphone as described above in certain embodiments) and/or ratios (i.e. the ratios of oxycodone:naloxone and hydromorphone:naloxone as described above in certain embodiments) and salts thereof as set out above.

It therefore needs to be understood that all embodiments described above referring to prolonged release dosage forms also describe corresponding further embodiments, wherein said dosage forms are immediate release dosage forms.

If an immediate release dosage form comprises oxycodone and naloxone (or salts thereof) is used, it can be particularly preferred that said dosage form comprises oxycodone or a pharmaceutically acceptable salt thereof in a ratio of about 2:1 to naloxone or a pharmaceutically acceptable salt thereof.

In a further preferred embodiment, the dosage form according to the invention comprises the opioid agonist and the opioid antagonist as the sole pharmaceutically active agents. The dosage form may be a prolonged release or an immediate release dosage form.

However, in another preferred embodiment, the dosage form according to the invention may comprise at least one further pharmaceutically active agent providing a further desired pharmaceutical effect in addition to the two active agents, i.e. the opioid agonist and the opioid antagonist. The dosage form may be a prolonged release or an immediate release dosage form.

In a further preferred embodiment, the prolonged release pharmaceutical dosage form comprises a prolonged release matrix in order to achieve the prolonged release.

In another preferred embodiment, the prolonged release dosage form comprises a prolonged release coating in order to achieve the prolonged release of the active agents.

In a further preferred embodiment, the prolonged release dosage form is an osmotic prolonged release dosage form.

When referring to a prolonged release matrix dosage form, the matrix preferably comprises a fatty alcohol and/or a hydrophobic polymer such as an alkylcellulose with ethylcellulose being particularly preferred.

Furthermore, in an also preferred embodiment, the dosage form may comprise further pharmaceutically acceptable ingredients and/or adjuvants, such as e.g. lubricants, fillers, binders, flowing agents, colourants, flavourants, surfactants, pH-adjusters, anti-tacking agents and/or combinations thereof. The dosage form may be a prolonged release or an immediate release dosage form.

In another preferred embodiment, the dosage form is an oral dosage form. However, the dosage form may also be a transdermal dosage form such as an immediate and/or sustained release skin patch.

In an also preferred embodiment, the dosage form is selected from the group comprising a tablet, a capsule, a multiparticulate, a dragee, a granulate, a liquid and a powder. A particularly preferred dosage form is a tablet or a multi-particulate.

If the patient suffers from delayed gastric emptying as symptom of Parkinson's disease, it can be preferred to use transdermal or liquid dosage forms according to the present invention.

In a further preferred embodiment, the at least one symptom of Parkinson's disease as referred to above is selected from a motor symptom including dyskinesia, hypokinesia, rigor (which may also be referred to as rigidity) and tremor; and a non-motor symptom (NMS) including disturbed gastrointestinal function such as delayed gastric emptying, constipation and disturbed bowel function, disturbed urogenital function such as urgency and nocturnia, cardiovascular symptoms, sleeping disorder, fatigue, apathy, drooling of saliva, difficulties in maintaining concentration, skin disorders, psychiatric disorders such as depression and anxiety, respiratory symptoms, cough, dyspnea and pain.

If the at least one symptom of Parkinson's disease is pain, such pain can be selected from musculoskeletal pain, radicular neuropathic pain, central neuropathic pain, dystonic pain, (Parkinson's disease related) chronic pain, fluctuation-related pain, nocturnal pain, coat-hanger pain, oro-facial pain and peripheral limb or abdominal pain, all of which are classified as being specifically related to or associated with PD. Pain may be observed in an “on”-phase, “off”-phase or in fluctuations.

In a further preferred embodiment, the present invention relates to a pharmaceutical dosage form for use in the treatment of at least one symptom of Parkinson's disease selected from dyskinesia, pain and constipation. Thus, the dosage form may be for use in the treatment of dyskinesia, which optionally may be induced by L-dopa treatment or another dopaminergic treatment such as dopamine agonist treatment. Additionally or alternatively, the dosage form may be for use in the treatment of pain and/or constipation as symptoms of Parkinson's disease. The dosage form may be a prolonged release or an immediate release dosage form.

In still another preferred embodiment, the present invention relates to a pharmaceutical dosage form for use in the treatment of pain in patients suffering from Parkinson's disease. Thus, the dosage form may be for use in the treatment of pain in patients with Parkinson's disease. Preferably, the dosage form may be for use in the treatment of moderate to severe pain in patients with Parkinson's disease. Thus, said pain may be due to Parkinson's disease and/or a symptom thereof in the Parkinson's disease patient population, and/or due to at least one further disease, the Parkinson disease patient is suffering from, such as e.g. cancer. The dosage form may be a prolonged release or an immediate release dosage form.

A “Parkinson's disease patient” or a “patient suffering from Parkinson's disease” as referred to herein has been diagnosed with Parkinson's disease according to any standard medical diagnostic criteria, e.g. the “UK Parkinson's disease society brain bank clinical diagnostic criteria” according to Hughes et al., JNNP 1992; 55:181-184. Such a patient may then be treated with a pharmaceutical preparation according to the invention for Parkinson's disease and/or a symptom thereof.

A dosage form according to the present invention may particularly be used in Parkinson's disease patients suffering from dyskinesia. Dyskinesia may be the most prominent symptom of Parkinson's disease in said patients.

In a particularly preferred embodiment, a dosage form according to the present invention may be used in Parkinson's disease patients suffering from L-Dopa induced dyskinesia (LID). LID may be the most prominent side effect of L-Dopa treatment in said patients. In this situation, the PD patients may still be treated with L-Dopa but may additionally be treated with a dosage form according to the present invention in order to treat the dyskinesia induced by L-Dopa. In another embodiment, such patients may be completely switched to a dosage form according to the present invention.

In a further particularly preferred embodiment, a dosage form according to the present invention may be used in Parkinson's disease patients suffering from dyskinesia induced by a dopaminergic. Dyskinesia induced by a dopaminergic may be the most prominent side effect of the dopaminergic treatment in said patients. In this situation, the PD patients may still be treated with a dopaminergic but may additionally be treated with a dosage form according to the present invention in order to treat the dyskinesia. In another embodiment, such patients may be completely switched to a dosage form according to the present invention.

In a particularly preferred embodiment, a dosage form according to the present invention may be used in Parkinson's disease patients suffering from dyskinesia induced by a dopamine agonist. In this situation, the PD patients may still be treated with a dopamine agonist but may additionally be treated with a dosage form according to the present invention in order to treat the dyskinesia.

In a particularly preferred embodiment, a dosage form according to the present invention may be used in Parkinson's disease patients suffering from dyskinesia induced by L-DOPA in combination with benserazide or carbidopa. In this situation, the PD patients may still be treated with L-DOPA/benserazide or L-DOPA/carbidopa but may additionally be treated with a dosage form according to the present invention in order to treat the dyskinesia.

A dosage form according to the present invention may also be used in Parkinson's disease patients, wherein said patients have not received opioid treatment before.

In a further preferred embodiment, a dosage form according to the present invention may be used in Parkinson's disease patients suffering from pain associated with Parkinson's disease.

In a further preferred embodiment, a dosage form according to the present invention may be used in Parkinson's disease patients suffering from pain associated with Parkinson's disease, wherein said pain cannot be treated in said patients by further increasing the dose of a dopaminergic since this increase would concurrently result in a worsening of the side effects due to the dopaminergic. Thus, said patients may already be treated with a dopaminergic but still suffer from pain to a degree, where further pain treatment is required; said treatment can be achieved by a dosage form according to the present invention.

A dosage form according to the present invention may particularly be used in Parkinson's disease patients suffering from pain associated with Parkinson's disease, wherein said pain cannot be treated in said patients by further increasing the dose of a dopaminergic since this increase would concurrently result in a worsening of the side effects due to the dopaminergic to a degree where therapy with the dopaminergic would need to be discontinued.

In a further preferred embodiment, a dosage form according to the present invention may be used in Parkinson's disease patients suffering from pain, wherein said pain would not be treated by a dosage form comprising an opioid agonist in patients not suffering from Parkinson's disease.

In a particularly preferred embodiment, a dosage form according to the present invention may be used in Parkinson's disease patients suffering from pain induced by dyskinesia as a symptom of Parkinson's disease or by dyskinesia induced by a dopaminergic. In this specific patient population, a dosage form according to the present invention may be used to treat dopaminergic-induced pain while simultaneously treating dopaminergic-induced dyskinesia.

In a particularly preferred embodiment, a dosage form according to the present invention may be used in Parkinson's disease patients suffering from pain induced by LID (as a side effect of L-Dopa treatment of Parkinson's disease). In this specific patient population, a dosage form according to the present invention may be used to treat LID-induced pain while simultaneously treating LID.

In a preferred embodiment, a dosage form according to the present invention may be used in Parkinson's disease patients suffering from musculoskeletal pain and/or radicular neuropathic pain and/or central neuropathic pain and/or dystonic pain and/or chronic pain and/or fluctuation-related pain and/or nocturnal pain and/or coat-hanger pain and/or oro-facial pain and/or peripheral limb or abdominal pain, wherein all of said pain types are PD-related and may be chronic.

A dosage form according to the present invention may also be used in Parkinson's disease patients suffering from constipation as symptom of Parkinson's disease. In such PD patients, constipation may be due to motor problems (e.g. the inability to control muscle contractions) and/or may be a consequence of lesions of the autonomic nervous system; however, the constipation is not due to treatment with an opioid agonist. Thus, said patients may also be defined as patients suffering from constipation as a symptom of PD, wherein said patients have not received opioid treatment before.

It can be particularly preferred to administer a dosage form according to the present invention to Parkinson's disease patients suffering from pain and constipation as defined above. It can further be particularly preferred to administer a dosage form according to the present invention to Parkinson's disease patients suffering from pain and dyskinesia as defined above. It can also be preferred to administer a dosage form according to the present invention to Parkinson's disease patients suffering from constipation and dyskinesia as defined above. It can be preferred to administer a dosage form according to the present invention to Parkinson's disease patients suffering from pain, constipation and dyskinesia as defined above.

In a particularly preferred embodiment, a dosage form according to the present invention may be used in PD patients undergoing treatment with a dopaminergic (or combinations thereof, such as L-DOPA and benserazide or L-DOPA and carbidopa) but still suffer from PD or symptoms of PD (such as pain or dyskinesia or constipation) to such a degree that further treatment is required, wherein a further increase in the dose of the dopaminergic is not possible due to an increase of side effects associated therewith. Such patients may thus be treated with a dopaminergic and a dosage form according to the present invention.

As mentioned above, the present invention in another object also relates to the use of an opioid agonist and an opioid antagonist in a pharmaceutical dosage form for the treatment of Parkinson's disease and/or at least one symptom thereof. The dosage form may be a prolonged release or an immediate release dosage form.

In this object, the agonist may in a preferred embodiment be selected from the group comprising morphine, oxycodone, hydromorphone, dihydroetorphine, etorphine, nalbuphine, propoxyphene, nicomorphine, dihydrocodeine, diamorphine, papavereturn, codeine, ethylmorphine, phenylpiperidine, methadone, dextropropoxyphene, buprenorphine, pentazocin, tilidine, tramadol, tapentadol, hydrocodone and pharmaceutically acceptable salts thereof. The opioid antagonist used in combination with the opioid agonist may preferably be selected from the group comprising naltrexone, naloxone, nalmefene, nalorphine, nalbuphine, naloxonazinene, methylnaltrexone, ketylcyclazocine, norbinaltorphine, naltrindol and pharmaceutically acceptable salts thereof.

In a preferred embodiment, oxycodone or a pharmaceutically acceptable salt thereof and naloxone or a pharmaceutically acceptable salt thereof are used in a pharmaceutical dosage form for the treatment of Parkinson's disease and/or at least one symptom thereof. The dosage form may be a prolonged release or an immediate release dosage form.

In another preferred embodiment, hydromorphone or a pharmaceutically acceptable salt thereof and naloxone or a pharmaceutically acceptable salt thereof are used in a pharmaceutical dosage form for the treatment of Parkinson's disease and/or at least one symptom thereof. The dosage form may be a prolonged release or an immediate release dosage form.

In other preferred embodiments, the opioid agonist and the opioid antagonist may be used in a pharmaceutical dosage form for the treatment of Parkinson's disease and/or at least one symptom thereof with specific active agents, combinations of said two active agents, corresponding amounts and/or ratios, salts thereof and so on as set out above in the first aspect relating to the dosage forms. The dosage form may be a prolonged release or an immediate release dosage form.

In a further preferred embodiment, the opioid agonist and the opioid antagonist are used in a pharmaceutical dosage form for treating at least one symptom of Parkinson's disease selected from pain, constipation and a dyskinesia, wherein the dyskinesia is optionally a LID. The dosage form may be a prolonged release or an immediate release dosage form.

### BRIEF DESCRIPTION OF THE FIGURES

FIG. 1 shows the study diagram of study I described in example 1.

FIG. 2 shows the schedules of visits in study I described in example 1.

FIG. 3 shows the treatments of the different phases in study I (3A: pre-randomisation run-in phase (open-label), treatment, dose and mode of administration; 3B: double-blind phases, test treatment, dose and mode of administration; 3C: double-blind phase study, reference treatment, dose and mode of administration).

FIG. 4 shows the subject's disposition in study I (randomised subjects).

FIG. 5 shows the disposition of subjects in study I.

FIG. 6 shows the study diagram of study II described in example 2.

FIG. 7 shows the schedules of visits and procedures in study II described in example 2.

FIG. 8 shows the treatments of the different phases in study II (8A: OxyIR-use during phases; 8B: double-blind treatment, double-blind phase; treatment, dose and mode of administration; 8C: open-label treatment; extension phase; treatment, dose and mode of administration; 8D: double-blind treatment; double-blind phase; treatment, dose and mode of administration).

FIG. 9 shows the subject's disposition in the double-blind safety population of study II.

FIG. 10 shows the disposition of subjects in study II.

FIG. 11 shows the study design of the study described in example 4.

FIG. 12 shows the screening of the study population at visit one of example 4 (referred to as Table 1 in example 4).

FIG. 13 shows the schedule of visits from randomisation to the end of the study described in example 4 (referred to as Table 2 in example 4).

### DETAILED DESCRIPTION OF THE INVENTION

The present invention partially resides in the surprising finding that a pharmaceutical dosage form comprising an opioid agonist and an opioid antagonist can be used in the treatment of Parkinson's disease and/or at least one symptom thereof, particularly for LIDs, pain and constipation.

### DEFINITIONS

Before some of the embodiments of the present invention are described in more detail, the following definitions are introduced.

As used in the specification and the claims, the singular forms of “a” and “an” also include the corresponding plurals unless the context clearly dictates otherwise. Thus, e.g. the term “dyskinesia” may also refer to “dyskinesias”.

The terms “about” and “approximately” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

In the context of the present, the term “prolonged release” refers to pharmaceutical compositions showing a slower release of the active agents than that of a conventional release pharmaceutical composition administered by the same route. Prolonged release is achieved by a special formulation design and/or manufacturing method. In general, “prolonged release dosage forms” in the context of the present invention means that the opioid agonist and the opioid antagonist are released from the pharmaceutical dosage form over an extended period of time.

The term “immediate release” as used herein refers to pharmaceutical compositions showing a release of the active substances which is not deliberately modified by a special formulation design and/or manufacturing methods. This will be set out in detail further below.

For purposes of the present invention, the term “opioid agonist” is interchangeable with the term “opioid analgesic” and includes one agonist or a combination of more than one opioid agonist; a partial agonist; stereoisomers thereof; an ether or ester thereof; or a mixture of any of the foregoing.

Opioid agonists useful in the present invention include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphine, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dextropropoxyphene, dihydroetorphine, fentanyl and derivatives, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papavereturn, pentazocine, phenadoxone, phenomorphan, phenazocine, phenoperidine, phenylpiperidine, piminodine, piritramide, propheptazine, promedol, properidine, propoxyphene, sufentanil, tilidine, tramadol, tapentadol, pharmaceutically acceptable salts, hydrates and solvates thereof, mixtures of any of the foregoing, and the like.

As used herein, the term “opioid antagonist” includes one antagonist or a combination of more than one opioid antagonist. Opioid antagonists generally counteract the effect of opioid agonists.

Opioid antagonists in accordance with the present invention may be selected from the group comprising naloxone, methylnaltrexone, alvimopan, naltrexone, methylnaltrexone, nalmemefe, nalorphine, nalbuphine, naloxonazine, ketylcyclazocine, norbenaltorphimine, naltrindole, 6-β-naloxole and 6-β-naltroxone, pharmaceutically acceptable salts, hydrates and solvates thereof, mixtures of any of the foregoing, and the like. It can be preferred to use an opioid antagonist having a low oral bioavailability such as naloxone.

It should be noted that nalorphine and nalbuphine are listed among both the opioid agonists and the opioid antagonists since both compounds exhibit agonistic as well as antagonistic properties. Thus, both nalorphine and nalbuphine act at the kappa receptors in an agonistic way, whereas they act on the mu-receptors in an antagonistic way.

If reference is made to an “opioid agonist” (such as e.g. oxycodone) or an “opioid antagonist” (such as e.g. naloxone), this always also includes the reference to a pharmaceutically acceptable salt of the free base of this pharmaceutically active agent unless it is specifically indicated that the reference to the pharmaceutically active agent should only refer to the free base.

Pharmaceutically acceptable salts include, but are not limited to, inorganic acid salts such as hydrochloride, hydrobromide, hydroiodide, sulfate, bisulfate, phosphate and the like; organic acid salts such as formate, acetate, trifluoroacetate, malate, maleate, tartrate, bitartrate, fumerate, succinate, citrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

“Parkinson's disease” as referred to herein refers to the generally accepted definition of this disease in the medical field. Thus, Parkinson's disease (PD) is a neurodegenerative disease, which can be characterized by motor symptoms and non-motor symptoms. Motor symptoms mainly include dyskinesia, hypokinesia, rigor and tremor, wherein hypokinesia includes bradykinesia and even akinesia. Non-motor symptoms include amongst others pain, constipation, delayed gastric emptying, depression and sleeping disorders. Due to the side effects of L-Dopa treatment, many PD patients also suffer from dyskinesias induced by L-Dopa (LIDs). Generally, patients treated with dopaminergics such as dopamine agonists may also suffer from dyskinesias. For the purpose of the present invention, LIDs or dopaminergic-induced dyskinesia may also be referred to as a symptom of PD.

“Treatment of Parkinson's disease” is to be understood as referring to a general improvement or even cure of the patient's PD-state or to the alleviation of PD. Such an improvement/cure or alleviation can either be detected by the patient's subjective feeling or by external observations.

“Treatment of a symptom of Parkinson's disease” is to be understood as referring to one or more specific symptom of PD, which can be improved, alleviated or even cured by a dosage form. Again, such an improvement, alleviation or cure can either be detected by the patient's subjective feeling or by external observations, particularly by clinical examination. As mentioned above, such symptoms may generally be divided into motor symptoms and non motor symptoms with the specific symptoms listed above. Clearly, more than one symptom may be improved by a dosage form such that it may be used in the treatment of at least one symptom of PD.

The term “dopaminergics” as used herein relates to substances commonly used in order to treat PD. This includes precursors of dopamine (such as L-DOPA), dopamine (receptor) agonists (such as lisuride and pergolide) and inhibitors of e.g. aromatic L-amino acid decarboxylase or DOPA decarboxylase (such as benserazide and carbidopa) as well as combinations thereof.

As mentioned inter alia in the “data collection and methods” section of the PRIAMO study by Barone et al (see above, page 1642 to 1643), there are specific scales and methods of assessment in order to evaluate PD and the symptoms thereof, e.g. for the assessment of the non motor symptoms. Thus, validated scales and methods exist, by which it can be assessed whether there is e.g. an improvement with respect to non motor symptoms (using e.g. a validated NMS questionnaire for PD consisting of 30 items in nine different domains (NMSQuest [see introductory part of the PRIAMO-study] or a validated questionnaire consisting of 12 NMS domains as mentioned in the data collection off the PRIAMO-study), whether the motor disability improved (using e.g. the unified PD rating scale part III, UPDRS-III) or whether the quality of life improved (using e.g. the 39 item PD Questionnaire, PDQ-39).

The inventors have surprisingly found that a dosage form comprising an opioid agonist and an opioid antagonist can particularly be used for the treatment of LID, pain and/or constipation.

Regarding pain it needs to be understood that pain may be a symptom of PD (e.g. so called “off-associated” pain which is not due to dyskinesia) and/or induced by a dyskinesia, particularly a LID as side effect of PD treatment as set out above.

Regarding constipation it needs to be understood that constipation may be a PD symptom (as outlined above it is even discussed as symptom preceding PD) and/or may be a side effect of an active agent used for the treatment of PD. Thus, if the constipation corresponds to a symptom of PD, which may precede PD, said constipation is not related to or induced by active agents such as e.g. opioid agonists. Nevertheless, it may be alleviated by a dosage form according to the present invention. However, if e.g. pain as symptom of PD and/or induced by LID is treated with an opioid analgesic, this can be frequently accompanied by the occurrence of constipation as side effect of the opioid analgesic. Clearly, this side effect, which might even be responsible for the worsening of an already existing constipation in a PD patient, should be avoided and the constipation should be alleviated. This may be achieved by administering a dosage form of the present invention comprising an opioid agonist and an opioid antagonist.

### Release Behaviour of the Dosage Form

In general, the release behavior of a dosage form can inter alia be determined by an in vitro release test.

In this regard, the term “in vitro release” refers to the release rate at which a pharmaceutically active agent, e.g. oxycodone HCl, is released from the pharmaceutical composition when the in vitro release rate is tested by the paddle method according to the European Pharmacopeia as described in the Ph. Eur. 2.9.3 6^{th} edition. The paddle speed is set at 100 rpm in simulated gastric fluid (SGF) dissolution medium with pH 1.2. Aliquots of the dissolution media are withdrawn at the respective time points and analyzed by HPLC with a C18 column, eluted with 30 mM phosphate buffer in acetonitrile (70:70; pH 2.9) with a flow rate of 1.0 ml/min and detected at 220 nm. The term “Simulated Gastric Fluid, pH 1.2” refers to 0.1 N HCl, pH 1.2. Usually, the mean value of six measurements is given for a specific release at a specific time point.

In contrast to an “immediate release”, a “prolonged release” dosage form in accordance with the present invention refers to pharmaceutical compositions which release in vitro ≦75% (by weight) of the pharmaceutically active agents, namely the opioid agonist and the opioid antagonist, at 45 min.

In the context of the present invention, the term “immediate release” refers to pharmaceutical compositions showing a release of the active substance(s) which is not deliberately modified by a special formulation design and/or manufacturing methods. For oral dosage forms this means that the dissolution profile of the active substance(s) depends essentially on its (theirs) intrinsic properties. Typically, the term “immediate release” refers to pharmaceutical compositions which release in vitro >75% (by weight) of the pharmaceutically active agent(s) at 45 min.

Prolonged release properties may be obtained by different means such as by a coating which is then designated as a prolonged release coating, a matrix which is then designated as a prolonged release matrix or e.g. by an osmotic structure of the pharmaceutical composition.

In order to obtain “prolonged release” properties, one typically uses materials which are known to prolong the release from a dosage form comprising e.g. a prolonged release matrix and/or prolonged release coating. Typical examples are set out further below. The nature of the “prolonged release material” may depend on whether the release properties are attained by a “prolonged release matrix” or a “prolonged release coating”. The term “prolonged release materials” thus describes both types of materials. The term “prolonged release matrix material” indicates that a material is used for obtaining a prolonged release matrix. Likewise, the term “prolonged release coating material” indicate that a material is used for obtaining a prolonged release coating.

The term “prolonged release matrix formulation” refers to a pharmaceutical composition including at least one prolonged release material, and at least the opioid agonist and the opioid antagonist as the two pharmaceutically active agents. In a “prolonged release matrix formulation”, the “prolonged release materials” are combined with the pharmaceutically active agents to form a mixture from which the pharmaceutically active agents are released over prolonged periods of time, such as e.g. 8, 10, 12, 14, 16, 18, 20, 22 or 24 hours.

It is to be understood that a material will be considered to act as prolonged release material if the dissolution profile of the pharmaceutically active agents is slowed down compared to an immediate or conventional release formulation. If a prolonged release material can be used for manufacturing a prolonged release matrix, it will be considered as a prolonged release matrix material.

Pharmaceutically acceptable excipients which are used to adjust an already prolonged release to a specific profile are not necessarily considered to be prolonged release materials.

It is to be understood that a prolonged release matrix does not necessarily consist only of the pharmaceutically active agents and the prolonged release material. The prolonged release matrix may comprise in addition pharmaceutically acceptable excipients such as fillers, lubricants, glidants, etc. Examples of such excipients are set out below.

The term “prolonged release coating formulation” refers to a pharmaceutical composition including at least one prolonged release material, and the opioid agonist and the opioid antagonist as the two pharmaceutically active agents. In a “prolonged release coating formulation”, the “prolonged release materials” are disposed on the pharmaceutically active agents to form a diffusion barrier. Other than in prolonged release matrix formulation, the actives are not intimately mixed with the prolonged release material and the prolonged release coating does not form a three dimensional structure within which the actives are distributed. As the term implies, the prolonged release material forms a layer above the actives. The pharmaceutically active agents are released from a prolonged release coating formulation over prolonged periods of time, such as e.g. 8, 10, 12, 14, 16, 18, 20, 22 or 24 hours.

It is to be understood that a material will be considered to act as prolonged release material if the dissolution profile of the pharmaceutically active agents is slowed down compared to an immediate or conventional release formulation. If a prolonged release material can be used for manufacturing a prolonged release coating, it will be considered as a prolonged release coating material.

Pharmaceutically acceptable excipients which are used to adjust an already prolonged release to a specific profile are not necessarily considered to be prolonged release materials.

When it is mentioned that a prolonged release coating is disposed on pharmaceutically active agents, this is not to be construed as meaning that such a coating will necessarily be directly layered on such active pharmaceutically agents. Of course, if the pharmaceutically active agents, the opioid agonist and the opioid antagonist, are layered on a carries such as nu-Pareil beads, the coating may be disposed directly thereon. However, the pharmaceutically active agents may also be first embedded in a polymer layer or e.g. a prolonged release matrix. Subsequently the prolonged release coating may be disposed on e.g. granules which comprise a prolonged release matrix or on tablets which are made from such granules by compression for example.

A pharmaceutical composition with a prolonged release coating may be obtained by combining the pharmaceutically active agents with a carries such as non-Pareil beads and disposing a prolonged release coating on said combinations. Such coating may be made from polymers such cellulose ethers with ethyl cellulose being preferred, acrylic resins, other polymers and mixtures thereof. Such prolonged release coatings may comprise additional excipients such as pore-formers, binders and the like.

It is further to be understood, that the term “prolonged release matrix formulation” does not exclude pharmaceutical compositions with a prolonged release matrix and an additional prolonged release coating being disposed on the matrix. Likewise the term “prolonged release coating formulation” does not exclude pharmaceutical compositions with a prolonged release coating which is disposed on prolonged release matrix.

The term “prolonged release dosage form” refers to the administration form of a pharmaceutical composition of the present invention comprising the two pharmaceutically active agents, i.e. the opioid agonist and the opioid antagonist, in prolonged release form as e.g. in form of a “prolonged release matrix formulation”, in the form of a “prolonged release coating formulation”, combinations thereof or in other prolonged release formulations such as osmotic formulations. The terms “prolonged release matrix formulation” and “prolonged release dosage form” can be used interchangeably if the prolonged release dosage form consists essentially of the prolonged release matrix formulation. This means that a prolonged release dosage form can comprise in addition to the prolonged release matrix e.g. cosmetic coatings and pharmaceutically acceptable excipients such fillers, lubricants, etc.

For some embodiments, the term “prolonged release matrix dosage form” may indicate that the dosage form comprises a prolonged release matrix as the sole structure being responsible for prolonging the release. This, however, does not exclude that the dosage form may comprise an immediate release portion.

For some embodiments, the term “prolonged release coating dosage form” may indicate that the dosage form comprises a prolonged release coating as the sole structure being responsible for prolonging the release. This, however, does not exclude that the dosage form may comprise an immediate release portion.

The release rates indicated always refer to the formulation such as a monolithic tablet or multi-particulates. The release rates will be chosen such that a pharmaceutical composition can be administered e.g. on a twice a day or once a day basis, i.e. every 12 hours or every 24 hours. Typically, the release will occur by diffusion through the prolonged release matrix and/or coating, erosion of the prolonged matrix and/or coating or combinations thereof.

The term “substantially equal release rate” as used herein means that the two active agents, i.e. the opioid agonist and the opioid antagonist (or salts thereof) are released from the dosage form such that their % of release does not deviate by more than about 20%, preferably by not more than about 15% and most preferably by not more that about 10%. In the most preferred embodiment, i.e. in the about 10% range, this means for example for a prolonged release dosage form comprising oxycodone and naloxone that if about 20% of oxycodone or a pharmaceutically acceptable salt are released from the dosage form in vitro after 15 minutes, naloxone will be released within a range of about 10% to about 30%, most preferably also at about 20% at 15 minutes.

### Release Materials

The following description of suitable materials is to be understood as being not limiting. Rather, the release material may be any material that is known to be capable of imparting prolonged release properties on the active agents, the opioid agonist and the opioid antagonist, when being formulated into a dosage form.

### Prolonged Release Matrix Materials

Suitable materials for inclusion in a prolonged release matrix in order to provide a prolonged release matrix dosage form comprising an opioid agonist and an opioid antagonist include:
(a) Hydrophilic or hydrophobic polymers, such as gums, cellulose ethers, acrylic resins and protein derived materials. Of these polymers, the cellulose ethers, especially alkylcelluloses are preferred. The dosage form may conveniently contain between 1% and 80% (by weight) of one or more hydrophilic or hydrophobic polymers.
(b) Substituted or unsubstituted hydrocarbons, such as fatty acids, fatty alcohols, glycerol esters of fatty acids, oils, and waxes. Hydrocarbons having a melting point of between 25 and 90° C. are preferred. The hydrocarbons may be long chain (C₈-C₅₀, preferably C₁₂-C₄₀) hydrocarbons. The hydrocarbons may be digestible. The oils and waxes may be vegetable, animal, mineral or synthetic oils and waxes. Of these hydrocarbon materials, fatty (aliphatic) alcohols are preferred. The dosage form may conveniently contain up to 60% (by weight) of at least one digestible, long chain hydrocarbon.
(c) Polyalkylene glycols. The dosage form may suitably contain up to 60% (by weight) of one or more polyalkylene glycols.

In a preferred embodiment, the pharmaceutical dosage forms as described in the present invention will use a diffusion matrix for achieving prolonged release of the opioid agonist and the opioid antagonist from the pharmaceutical dosage form.

To this end, the diffusion matrix may be made from a hydrophobic polymer and/or a C₁₂-C₃₆ fatty alcohol.

As regards the hydrophobic polymer, use of a hydrophobic cellulose ether and particularly ethyl cellulose may be preferred.

As regards the fatty alcohol, use of lauryl, myristyl, stearyl, cetylstearyl, ceryl and/or cetylalcohol will be preferably considered. The use of stearyl alcohol is particularly preferred.

A particularly preferred embodiment relates to pharmaceutical dosage forms in which the prolonged release properties of the opioid agonist and the opioid antagonist are provided by a diffusion matrix which is made from a hydrophobic polymer such as from ethyl cellulose and a fatty alcohol. The matrices of some of the preferred embodiments of the invention, which may e.g. be made from the aforementioned combination of ethyl cellulose and stearyl alcohol, will be a substantially non-swellable diffusion matrix.

The term “substantially non-swellable diffusion matrix” indicates that the matrix will be substantially non-erosive, i.e. that the size of the matrix will not significantly increase upon contact with fluids. Typically, the volume of a substantially non-swellable diffusion matrix will increase at maximum up to 100%, preferably at maximum up to 75%, more preferably at maximum up to 50%, even more preferably at maximum up to 25% and most preferably at maximum up to 10% or at maximum up to 5% in volume upon contacting an aqueous solution.

Pharmaceutical dosage forms which comprise a hydrophobic polymer with hydrophobic cellulose ethers such as ethyl cellulose being preferred as the sole or one of the components for providing a prolonged release (non-swellable) diffusion matrix, will use an amount of such polymer of between 5 to 20%, preferably of between 6 and 15% by weight and more preferably of between 7 to 10% by weight. The percentages indicate the amount of the matrix-forming material with respect to the total weight of the pharmaceutical dosage form.

Pharmaceutical dosage forms, which comprise a fatty alcohol as the sole or one of the components for providing a prolonged release diffusion matrix, will use an amount of fatty alcohol in the matrix of between 10 to 40%, preferably of between 15 to 35% and more preferably of between 17 to 25% by weight. These percentages again indicate the amount of fatty alcohol based on the total weight of the dosage form.

The person skilled in the art is further aware that such a prolonged release matrix may also contain other pharmaceutically acceptable ingredients and excipients which are conventional in the pharmaceutical art such as lubricants, fillers, binders, flowing agents, colourants, flavourants, surfactants, pH-adjusters, anti-tacking agents and granulating aids. These excipients will typically have no substantial impact on the overall release behavior of the pharmaceutical dosage form.

Typical examples of fillers (diluents) comprise lactose, preferably anhydrous lactose, glucose, saccharose, starch and their hydrolysates, microcrystalline cellulose, cellatose, sugar alcohols such as sorbitol or mannitol, calcium salts like calcium hydrogen phosphate, dicalcium- or tricalcium phosphate. Granulating aids comprise inter alia povidone. Flowing agents and lubricants comprise inter alia highly dispersed silica, talcum, magnesium oxide, calcium stearate, magnesium stearate, sodium stearyl fumarate, fast like hydrated castor oil and glyceryl dibehenate. Binders can include hyproxypropylmethyl cellulose (hypromellose), hydroxypropyl cellulose, hydroxyethyl cellulose, polyvinyl pyrollidone (povidone), acetic acid vinyl ester (copovidone) and carboxymethycellulose sodium. Anti-tacking agents may include glycerol monostearate. Furthermore, a matrix-based dosage form may e.g. comprise a cosmetic coating.

### Prolonged Release Coating Materials

As mentioned above, prolonged release characteristics of a pharmaceutical dosage form may also be achieved by a film coating that governs the release of the active agents from the dosage form. To this end, the pharmaceutical dosage form may comprise a carrier, which is associated with the opioid agonist and the opioid antagonist. For example, one may use nonpareil beads, sugar beads etc. on and/or into which the pharmaceutically active agents are disposed.

Such active-associated carriers may then be overcoated with a coating that provides prolonged release characteristics. Suitable prolonged release coating materials include hydrophobic polymers such as cellulose ethers and/or acrylic polymer resins. Ethylcellulose may be preferred.

The prolonged release coatings may comprise other components such as hydrophilic substances including hydrophilic polymers such hydroxypropylmethylcellulose (HPMC), polyethylenglycols etc. These components may be used to adjust the prolonged release characteristics of the coatings. In case of e.g. HPMC, the substances may act as pore formers. The coating may, of course, also comprise additional pharmaceutically acceptable excipients, e.g. as set out above for the matrices.

### Immediate Release Materials

Typical pharmaceutically acceptable excipients used in immediate release dosage forms are disintegrants, diluents, lubricants, glidants, anti-tacking agents, plasticizers, colourants, flavorants, binders, pH adjusters and the like. These excipients (with the exception of disintegrants) are to be chosen such that they do not substantially alter the immediate release in vitro release rates.

It can be preferred for the pharmaceutical compositions of the present invention to comprise at least a diluent and optionally a disintegrant as pharmaceutically acceptable excipients, particularly if the pharmaceutical compositions of the present invention are provided as a tablet. It can also be preferred for the pharmaceutical compositions of the present invention to comprise at least a disintegrant and optionally a diluent as pharmaceutically acceptable excipients, particularly if the pharmaceutical compositions of the present invention are provided as a tablet. It can further be preferred to use excipients which act both as a disintegrant and a diluent.

The disintegrant, for example, will ensure that the tablet after administration will rapidly disintegrate so that the active agents become readily available for absorption.

Diluents may be selected from but are not limited to lactose such as lactose monohydrate, lactose anhydrous, starch such as maize starch, pregelatinized starch, microcrystalline cellulose, glucose, Mannitol, Maltitol, StarLac® (85% spray dried lactose, 15% maize starch), saccharose, calcium salts like calcium hydrogen phosphate or any combinations of the above.

Disintegrants may be selected from but are not limited to inter alia StarLac® (85% spray dried lactose, 15% maize starch), croscarmellose such as croscarmellose sodium, sodium starch glycolate, crospovidone, alginic acid, or low substituted hydroxypropyl cellulose.

A combination of lactose and starch such as the Starlac® product can be particularly preferred as it combines the properties of a filler and a disintegrant.

Glidants and lubricants may be selected but are not limited to inter alia highly dispersed silica, talcum, magnesium oxide, magnesium stearate, sodium stearyl fumarate etc.

Flowing agents and lubricants comprise inter alia highly dispersed silica, talcum, magnesium oxide, magnesium stearate, sodium stearyl fumarate etc.

If pharmaceutical compositions of the present invention are provided as a tablet, they may be coated for identification purposes with a cosmetic coating. Such coatings will have no substantial impact on the immediate release properties of the pharmaceutical compositions in accordance with the invention.

Preferably, one can use a combination of e.g. starch and lactose as disintegrant. Lactose alone may at the same time function as a filler. A particularly preferred embodiment relies on the product Starlac®, a combination of lactose 85% and starch 15%, which may function both as a disintegrant and as a filler. The combined filler/disintegrant may be comprised within the pharmaceutical composition in an amount of about 40% to about 90%, preferably in an amount of about 50% to about 85% and even more preferably in an amount of about 60% to about 80% by weight based on the weight of the composition. These numbers particularly apply if an excipient having a dual function both as a disintegrant and a filler such as Starlac® is used.

The invention is now illustrated with respect to specific examples. These examples are, however, not to be construed as limiting.

### EXAMPLES

### Example 1

### Improvement of Constipation and Pain in PD Patients: STUDY I

### Objective:

The primary objective of study I was to demonstrate that subjects with moderate to severe non malignant pain taking OXN PR (oxycodone+naloxone in a prolonged release dosage form) have improvement in symptoms of constipation as measured by the BFI compared to subjects taking OxyPR (oxycodone in a prolonged release dosage form) alone. A secondary objective was to estimate the subjects'Average Pain over the last 24 Hours assessed at each double-blind study visit during treatment with OXN PR compared with OxyPR as measured by the Pain Intensity Scale. Three patients suffering from Parkinson's disease were among the subjects participating in the study.

### Overall Study Design and Plan:

Study I was a randomized (1:1 ratio), double-blind, double-dummy, parallel group, multicenter, 12-week study to demonstrate improvement in symptoms of constipation in subjects taking oxycodone equivalent of 60-80 mg/day as OXN PR compared to subjects taking OxyPR alone.

Subjects must have had non-malignant pain, which was being treated with an opioid analgesic and must have been experiencing constipation secondary to opioid treatment. A sufficient number of subjects were planned to be enrolled to randomize 266 subjects, with subjects randomized to OXN PR and OxyPR (133 subjects/group).

Three patients suffering from Parkinson's disease participated in the study; two of them were in the OxyPR group, whereas one PD patient was in the OXN PR group.

This study was composed of three phases: a Pre-randomisation Phase, a Double-blind Phase, and an Extension Phase. The core study was comprised of the Pre-randomisation Phase and Double-blind Phase. The Pre-randomisation Phase contained two periods: the Screening Period and the Run-in Period. The Screening Period involved prospective assessments and was designed to qualify subjects for participation in the Run-In Period. The Run-In Period was designed to titrate OxyPR to analgesic effect, to convert to the study laxative, to qualify subjects for participation in the Double-blind Phase, and enable identification of an effective dose for the study medication to be used after randomisation. The Double-blind Phase was designed to demonstrate the safety and efficacy of OXN PR versus OxyPR in producing improvement in symptoms of constipation secondary to opioid treatment of moderate to severe nonmalignant pain. Available to those subjects who completed the Double-blind Phase, the Extension Phase was designed to assess the long-term safety of OXN PR for up to 52 additional weeks.

Efficacy assessments were collected in daily diaries and during periodic visits. The Primary efficacy variable was the BFI. Secondary efficacy variables were the mean of the rectal and stool subscale scores of the PAC-SYM (PACOI), PAC-SYM(b), Patient Global Impression of Change (PGIC), and Pain Intensity Scale.

Safety was assessed using adverse events (AEs, learned through spontaneous reports, subject interview, or subject diaries), clinical laboratory results, vital signs, physical examinations, electrocardiograms (ECGs) and SOWS. Estimates of the population mean and population variability for oxycodone and naloxone PK parameters were derived using a nonlinear mixed effects model, i.e., a population PK approach, using up to 3 samples per subject.

To the treatments assigned in the double-blind phase of the study, both subjects and investigators were blinded. Also the sponsor personnel to be involved in the data processing and the statistical analysis for this study were blinded to treatment assignments. Treatments were masked in a double dummy fashion, whereby subjects to receive OXN PR were given OXN PR and OxyPR placebo, and subjects to receive OxyPR were given OxyPR and OXN PR placebo.

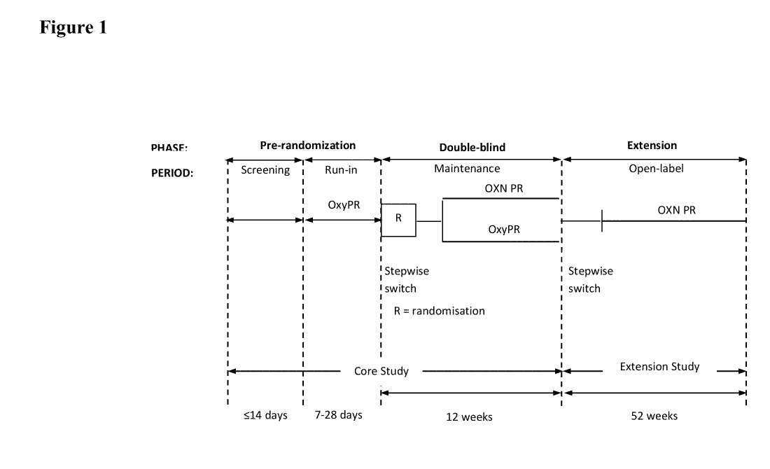

The corresponding study diagram is presented in FIG. 1.

### Pre-Randomisation Phase:

the Pre-randomisation Phase duration was up to 42 days. The Pre-randomisation Phase, containing a Screening Period and Run-in Period, was designed to (a) assess inclusion/exclusion criteria, (b) convert pre-study opioid therapy to open-label OxyPR and titrate to an effective analgesic dose of 60-80 mg OxyPR/day, (c) convert pre-study laxative therapy to the study laxative to be used per study routine for constipation, and (d) identify the dose of study medication to be used during the Double-blind Phase.

### Screening Period:

the screening period could last for up to 14 days. To be eligible to enter the Screening Period, subjects must have been at least 18 years of age and have a documented history of moderate to severe chronic nonmalignant pain that required around-the-clock opioid therapy (oxycodone equivalent of 60-80 mg/day). At Visit 1, subjects underwent complete evaluation for study eligibility (i.e., all inclusion/exclusion criteria) and those who qualified entered the Run-in Period.

### Run-in Period:

the Run-in Period lasted 7 to 28 days. At Visit 2, qualified subjects had their pre-study opioid therapy converted to open-label OxyPR, which was titrated to an effective analgesic dose. Qualified subjects also had their pre-study laxative therapy converted to bisacodyl 10 mg/day to be taken no sooner than 72 hours after their most recent BM as rescue medication for constipation. The 7-day baseline assessment in the Run-in Period started no sooner than the day of the initial dose conversion to OxyPR.

The initial starting dose of open-label OxyPR was calculated by converting a subject's total daily dose of their prior opioid to an oxycodone PR equivalent. The total daily oxycodone PR equivalent dose was divided by 2 and rounded to the nearest 10 mg to determine the q12 h doses. Subjects took open-label OxyPR every 12 hours. Asymmetric dosing was permitted only in the 70 mg/day OxyPR dosing group, where the AM and the PM doses were not identical. OxyIR was prescribed q4 h PRN. If a subject was consistently taking more than two OxyIR rescue doses/day for break-through pain, then the OxyPR medication was uptitrated. Subjects who required more than 80 mg of OxyPR for adequate analgesia during the Run-in phase were discontinued from the study.

Subjects were required to show they had obtained an effective analgesic dose for the last 7 days of the Run-in Period and had fewer than 3 CSBM-NS during this time (the baseline assessment).

After Visit 2, supplemental visits for titration to effective analgesia could be conducted.

Subjects who achieved adequate analgesia on an OxyPR dose between 60-80 mg/day and had confirmed opioid-related constipation were eligible to be randomised and to enter the Double-blind Phase. To continue in the study and enter the Double-blind phase, subjects must also have continued to meet all eligibility criteria and demonstrate compliance with taking open-label OxyPR and completing daily diaries.

The maximum duration of the Run-in Period (including the baseline assessment during which subjects have maintained a stable OxyPR dose) was 28 days. If after 28 days of the Run-in Period, the subject had not achieved stable pain control, was taking >80 mg OxyPR/day, did not have confirmed opioid-related constipation, or did not meet other inclusion/exclusion criteria, the subject did not enter the Double-blind Phase, was discontinued from the study and resumed his or her pre-study treatment, upon consultation with the investigator. If a subject discontinued from the study early (prior to Visit 8), then the end of study visit (Visit 8 assessments) were conducted as soon as possible after the decision is made to terminate participation.

### Double-Blind Phase:

the Double-blind Phase was 12 weeks in duration. At Visit 3, subjects who achieved stable pain control in the Run-In period and had confirmed opioid related constipation were randomised in a 1:1 ratio to the Double-blind study medication (i.e., OXN PR or OxyPR) every 12 hours.

Investigators provided instructions to the subjects regarding study medication and laxative dosing. Subjects were converted from the effective dose of OxyPR established during the Run-in Period to the equivalent dose (in mg of oxycodone prolonged release per day) of the double-blind study medication in a stepwise manner over a period of 4 days within the first week of the Double-Blind phase. Subjects took their first dose of double-blind study medication on the evening of Visit 3. Study medication dosing was q12 h with a fixed dose; the AM and PM doses could be symmetrical or asymmetrical (70 mg/day). Open-label OxyIR was provided as add-on therapy (i.e., rescue medication). OxyIR was prescribed q4 h PRN. If a subject was consistently taking more than two OxyIR rescue doses/day for break-through pain, then the oxycodone prolonged-release medication was uptitrated. If a dose above 80 mg oxycodone PR/day was needed, an uptitration in a double-dummy manner to 120 mg/day oxycodone PR during the Double-blind Phase was permitted (Subjects on 80 mg were titrated to 100 mg/day oxycodone prolonged release; subjects on 100 mg/day oxycodone PR were uptitrated to 120 mg/day oxycodone prolonged release).

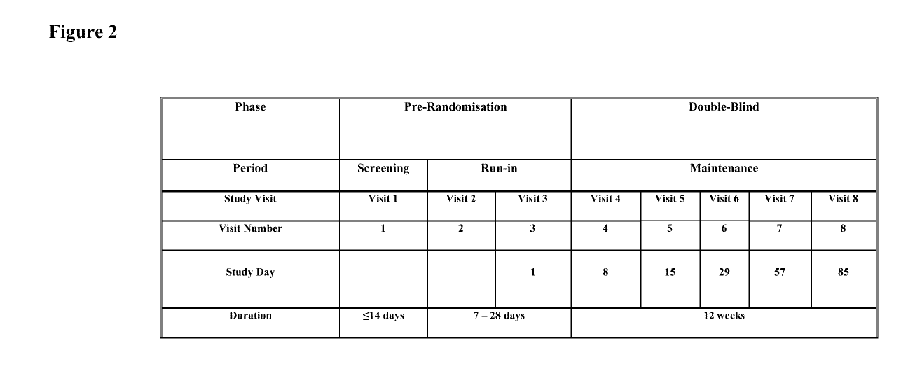

During the Double-blind Phase subjects were only permitted to take oral bisacodyl 10 mg/day 72 hours after their most recent BM as rescue medication for constipation. Other laxatives, except for fiber supplementation or bulking agents, were permitted. Subjects received double-blind study medication for approximately 12 weeks. Study visits occurred at Days 8, 15, 29, 57, and 85 with a ±3 days study window (see FIG. 2).

Subjects completed daily diaries to collect bowel function data, pain scores, and laxative use. Rescue medication use was recorded on the rescue medication blister card. Modified SOWS were completed daily in the diary during the first week of the double-blind phase. Modified SOWS were also collected at Visits 3, and 4.

Subjects returned at Visit 8 to complete End-of-Study procedures. A Treatment Satisfaction assessment was completed at this visit. Further visits to the study site were conducted if considered necessary for the subject's welfare.

Subjects who did not tolerate the study medication were discontinued from the study. Site study staff members discontinued the subject from the study and the subject returned to the clinic for appropriate therapy according to standard of care.

If a subject discontinued from the study early (prior to Visit 8), then the end of study visit (Visit 8 assessments) were conducted as soon as possible after the decision was made to terminate participation.

### Selection of Study Population:

subjects had moderate to severe chronic nonmalignant pain that required around-the-clock opioid therapy (oxycodone PR equivalent of 60-80 mg/day) and also had constipation secondary to opioid treatment. Approximately 266 subjects were to be randomised into the Double-blind Phase. An adequate number of subjects were to be screened in the Pre-randomisation Phase to achieve this sample size.

### Inclusion Criteria:

subjects who were included in the study were those who meet all of the following screening criteria:
Male or female subjects at least 18 years or older.
Female subjects less than one year post-menopausal must have had a negative serum pregnancy test recorded prior to the first dose of study medication, be non-lactating, and willing to use adequate and reliable contraception throughout the study.
Moderate to severe chronic nonmalignant pain that requires around-the-clock opioid therapy (oxycodone equivalent of 60-80 mg/day).
Subjects who required continuation of daily opioid treatment and were likely to benefit from WHO step III opioid therapy for the duration of the study.
Subjects must have been willing to discontinue their current opioid analgesic routine.
Subjects were to report constipation caused or aggravated by opioids.
Subjects had to be willing to discontinue their current laxative regimen.
Subjects had to comply with the use of oral bisacodyl as laxative rescue medication. Rescue was permitted no sooner than 72 hours after the subject's most recent bowel movement (BM).
Subjects taking daily fibre supplementation or bulking agents were eligible if they could be maintained on a stable dose and regimen throughout the study, and in the investigator's opinion were willing and able to maintain adequate hydration.
Subjects willing and able to participate in all aspects of the core study, including use of oral medication, completion of subjective evaluations, attending scheduled clinic visits, completing telephone contacts, and compliance with protocol requirements as evidenced by providing written, informed consent.
Subjects with pre-study, non-opioid analgesics, and all other concomitant medications, including those medications for the treatment of depression, that were thought to be stable, were considered necessary for the subject's welfare, were anticipated to remain stable throughout the Double-blind Period of the study, and were to be continued under the supervision of the investigator, were eligible.

### Exclusion Criteria:

subjects who were to be excluded from the study were those who meet any of the following screening criteria:
Females who were pregnant (positive β-hCG test) or lactating.
Any history of hypersensitivity to oxycodone, naloxone, or related products.
Any contraindication to bisacodyl.
Subjects with evidence of significant structural abnormalities of the gastrointestinal (GI) tract (e.g., bowel obstruction, strictures) or any diseases/conditions that affect bowel transit (e.g., ileus, hypothyroidism).
Subjects with cancer associated pain.
Active alcohol or drug abuse and/or history of opioid abuse.
Subjects with Rheumatoid Arthritis (RA).
Subjects with evidence of clinically unstable disease, as determined by medical history, clinical laboratory tests, ECG results, and physical examination that, in the investigator's opinion, precluded entry into the study.
Subjects with evidence of impaired liver/kidney function upon entry into the study defined as aspartate aminotransferase (AST; SGOT), alanine aminotransferase (ALT; SGPT), or alkaline phosphatase levels >3 times the upper limit of normal; gamma glutamyl transpeptidase (GGT or GGTP)≧5 times the upper limit of normal; total bilirubin level outside of the reference range; and/or creatinine level outside of the reference range or >2 mg/dl, or in the investigator's opinion, liver and/or kidney impairment to the extent that the subject should not participate in this study.
Subjects who have required treatment for the diagnosis of irritable bowel syndrome (IBS).
Subjects receiving hypnotics or other central nervous system (CNS) depressants that, in the investigator's opinion, may pose a risk of additional CNS depression with opioids study medication.
Subjects receiving opioid substitution therapy for opioid addiction (e.g., methadone or buprenorphine).
Subjects who participated in a clinical research study involving a new chemical entity or an experimental drug within 30 days of study entry (defined as the start of the Screening Period).
Subjects presently taking, or who had taken naloxone or naltrexone within 30 days of study entry (defined as the start of the Screening Period).
Surgery within 2 months prior to the start of the Screening Period, or planned surgery during the 12-week Double-blind Phase that could have affected GI motility or pain.
   Criteria for Entry into the Double-Blind Phase:

subjects who were included in the Double-blind Phase of the study were those who met all of the following screening criteria:
Subjects continued to satisfy Screening Inclusion/Exclusion criteria.
Subject's OxyPR dose was between 60-80-mg/day.
Subjects rated their pain (“average pain” over the last 24 hours) as ≦4 on 0-10 scale with less than or equal to two doses of oxycodone immediate release (OxyIR) rescue medication per day for either the last three consecutive days or four of the last seven days.
Subjects must have had confirmed opioid related constipation, which was defined as having less than 3 CSBM-NS during the last 7 days.
Subjects demonstrated compliance with laxative use, taking open-label OxyPR, and completing daily diaries.

### Schedule of Visits and Procedure:

FIG. 2 presents the schedule of visits and procedures for the study.

Efficacy assessments were collected in daily diaries and during periodic visits.

### Primary Efficacy Variable:

a subject's Bowel Function Index (BFI) score was the arithmetic mean of the following items (assessed at each visit):

1) Ease of defecation (numerical analogue scale [NAS], 0=easy/no difficulty; 100=severe difficulty); 2) Feeling of incomplete bowel evacuation (NAS, 0=not at all, 100=very strong); 3) Personal judgment of constipation (NAS, 0=not at all, 100=very strong). Each of the questions referred to the last 7 days for the subject.

### Secondary Efficacy Variable:

Pain Intensity Scale—Average Pain over the last 24 Hours, as assessed at each double-blind study visit (Scale of 0-10, 0=no pain; 10=strong pain).

### Treatments Administered:

study medication includes any drug(s) under evaluation in the study, including reference drug(s) and placebo but not including rescue medication. The dispensing of study medication and rescue medication could be adjusted during the study, either by the Investigator at individual sites after prior consultation with the Sponsor, or by the Sponsor for all sites, as needed to manage the risk of abuse or diversion. Subjects took their first dose of study drug at home at the time of their next regularly scheduled dose of medication.

The treatments administered in the study are presented in the following sections.

### Pre-Randomisation Run-in Period:

the Run-in Period of the Pre-randomisation Phase was designed to convert pre-study opioid therapy to open-label OxyPR and titrate to an effective analgesic dose (60-80 mg OxyPR/day), to convert pre-study laxative therapy to the study laxative to be used per study routine for constipation, and to identify the dose of study medication to be used during the Double-blind Phase.

The initial dose of open-label OxyPR was calculated by converting a subject's total daily dose of prior opioids to an oxycodone PR equivalent. The total daily oxycodone PR equivalent dose was divided by 2 and rounded to the nearest 10 mg to determine the q12 h doses. Subjects were to take open-label OxyPR every 12 hours. Asymmetric dosing was permitted only on the 70 mg/day dose as long as the maximum dose of oxycodone per day did not exceed 80 mg.

Subjects were permitted to take OxyIR for rescue; it could be dosed every 4 hours. If a subject was consistently taking more than two OxyIR rescue doses/day for break-through pain, then the oxycodone prolonged release medication was uptitrated. Subjects on 80 mg of OxyPR per day, who required more than 2 rescue doses of OxyIR for 3 consecutive days during the Run-in phase, were to be discontinued from the study.

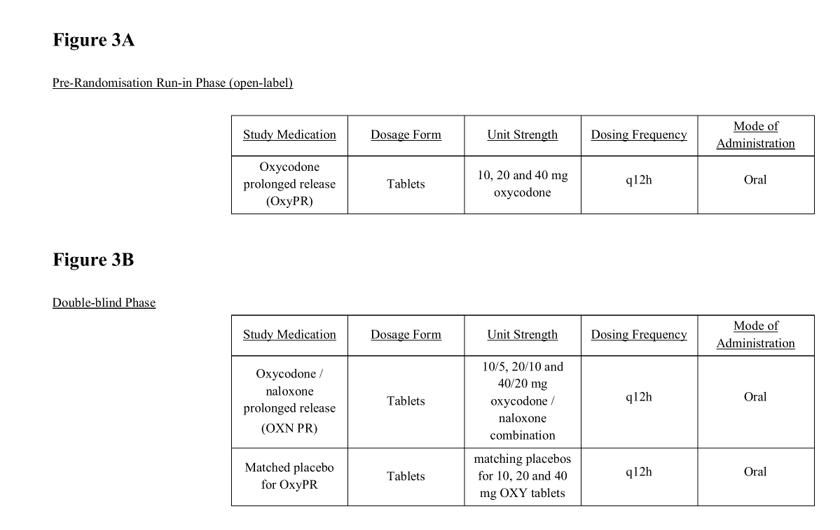

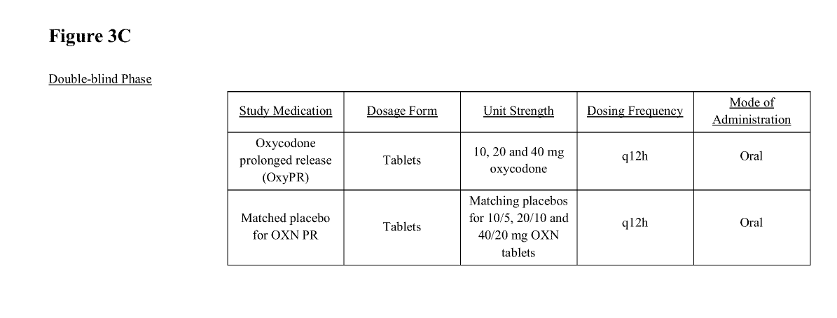

The Pre-Randomisation Run-in Phase is shown in FIG. 3A.

At Visit 2, the subject was dispensed 2 weeks worth of medication. If the subject required titration to a different dose of OxyPR the subject returned for an unscheduled visit. In addition, a medication resupply visit could be scheduled for 2 weeks after Visit 2. At this visit the subject was dispensed with a further 2 weeks of medication, if necessary.

### Double-Blind Phase:

Subjects started the Double-blind Phase at the same dose level (in mg oxycodone PR/day) that they received at the end of the Run-in Period. The switch to randomised double-blind study medication was done over a period of 4 days within the first week of the double-blind phase. The first dose of double blind study medication was the evening dose of Visit 3. Subjects received double-blind study medication for up to 12 weeks.

Subjects were permitted to take oxycodone immediate release (OxyIR) for rescue; it could be dosed every 4 hours. If a subject was consistently taking more than 2 OxyIR rescue doses/day for break-through pain, then the oxycodone prolonged release medication must be uptitrated. If a dose above 80 mg OxyPR/day was needed, a titration up to 120 mg/day OxyPR during the Double-blind Phase was permitted. The test treatment, dose, and mode of administration are shown in FIG. 3B. The reference treatment, dose, and mode of administration are shown in FIG. 3C.

### Subject Disposition:

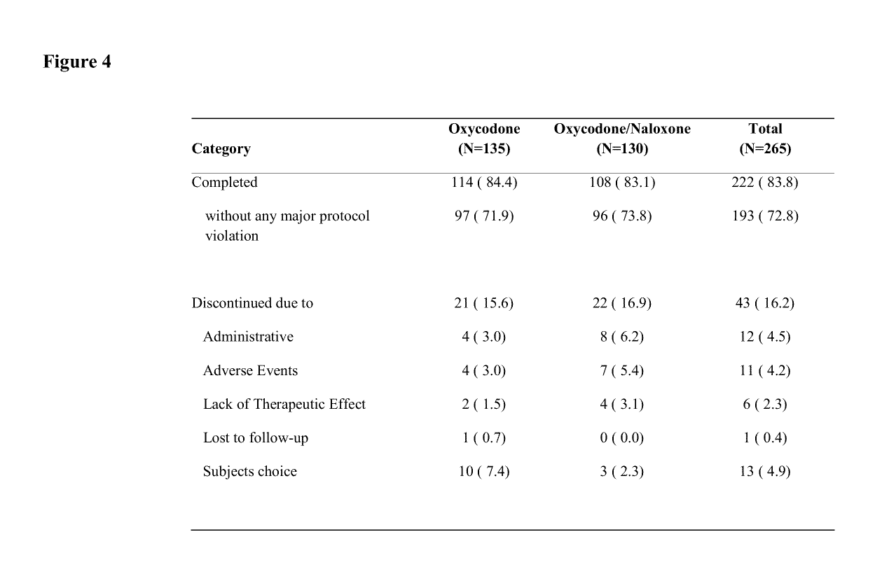

in total 379 subjects were screened for entry into the study, 32 subjects were screening failures and 347 subjects were enrolled, 331 subjects were entered in the safety run-in period and 278 subjects were randomised into the double-blind phase of the study. 135 subjects were randomised to receive OxyPR and 130 were randomised to receive OXN PR. FIG. 4 summarizes the disposition of the 265 subjects randomised to treatment by treatment group.

FIG. 4 shows all randomized subjects.

In total 222 subjects completed the study. Overall the discontinuation rate was low and similar in both treatment groups (15.6% in the OxyPR group, 16.9% in the OXN PR group). The main reason for early discontinuation was subjects choice (7.4%) in the OxyPR treatment group, and discontinuation due to administrative reasons in the OXN PR group (6.2%). The discontinuation rate due to AEs and administrative reasons was slightly higher in the OXN PR group compared to the OxyPR treatment group, whereas a slightly higher discontinuation rate due to subjects choice was documented in the OxyPR group.

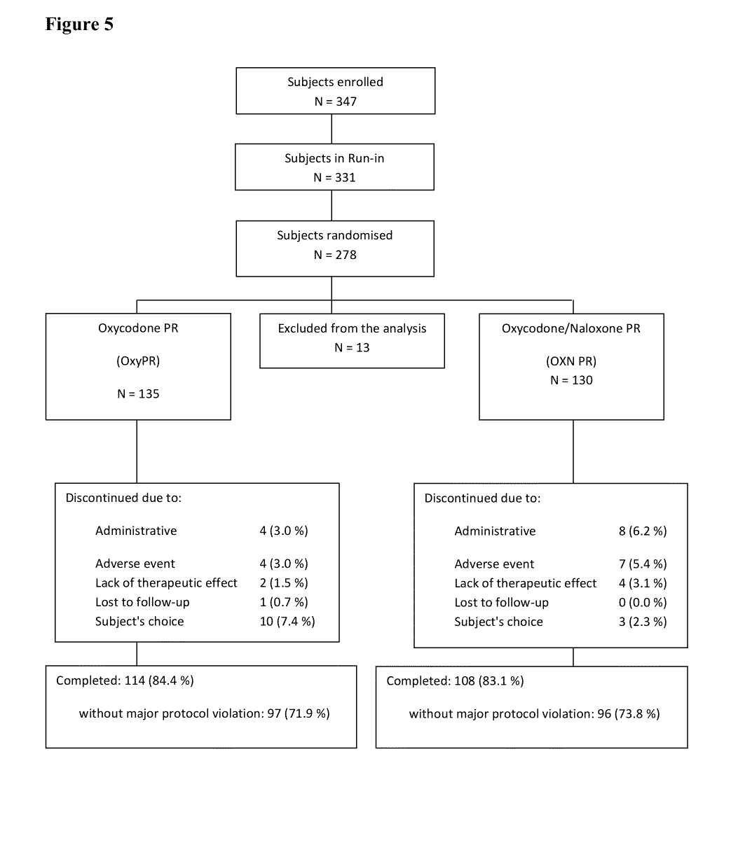

FIG. 5 displays the disposition of subjects in study I.

### Results for the Three PD-Patients:

As mentioned above, the BFI and the pain intensity (PI) were determined at each of Visits 1 to 8. One PD patient (subject “A”) received OXN for treatment, whereas the other two PD patients (subjects “B” and “C”) received OXY. Values in italic for subjects B and C indicate that the BFI and the PI were not determined but that the values of the previous visits were still applicable.

| Subject | Visit Number | BFI | PAIN | TRT |
|---|---|---|---|---|
| B | 1 | 85.00 | 3.00 | OXY |
| B | 2 | 83.33 | 2.00 | OXY |
| B | 3 | 76.67 | 1.00 | OXY |
| B | 4 | 73.33 | 0.00 | OXY |
| B | 5 | 73.33 | 0.00 | OXY |
| B | 6 | 73.33 | 0.00 | OXY |
| B | 7 | 73.33 | 0.00 | OXY |
| B | 8 | 73.33 | 0.00 | OXY |
| A | 1 | 53.33 | 3.00 | OXN |
| A | 2 | 53.33 | 3.00 | OXN |
| A | 3 | 36.67 | 3.00 | OXN |
| A | 4 | 23.33 | 3.00 | OXN |
| A | 5 | 16.67 | 3.00 | OXN |
| A | 6 | 6.67 | 3.00 | OXN |
| A | 7 | 10.00 | 2.00 | OXN |
| A | 8 | 3.33 | 3.00 | OXN |
| C | 1 | 63.33 | 4.00 | OXY |
| C | 2 | 55.00 | 3.00 | OXY |
| C | 3 | 70.00 | 4.00 | OXY |
| C | 4 | 50.00 | 3.00 | OXY |
| C | 5 | 50.00 | 5.00 | OXY |
| C | 6 | 80.00 | 4.00 | OXY |
| C | 7 | 80.00 | 4.00 | OXY |
| C | 8 | 86.67 | 4.00 | OXY |

### BFI-Score:

arithmetic mean of the following items:

1) Ease of defecation (numerical analogue scale [NAS], 0=easy/no difficulty; 100=severe difficulty); 2) Feeling of incomplete bowel evacuation (NAS, 0=not at all, 100=very strong); 3) Personal judgment of constipation (NAS, 0=not at all, 100=very strong).

### Pain-Score:

Average Pain on a 10-point ordinal scale, 0=no pain; 10=pain as bad as you can imagine.

### Example 2

### Improvement of Constipation and Pain in PD Patients: STUDY II

Objective with Respect to Pain:

To demonstrate the superiority of OXN over placebo on the time from the initial dose of study medication to multiple (i.e. recurring) pain events (inadequate analgesia) during the Double-blind Phase. A pain event was demonstrated by unacceptable pain control for 2 consecutive days. Each pain event was 2 discrete days, e.g. there could be a maximum of 2 pain events in 4 days.

Objective with Respect to the Bowel Function:

To determine the degree of constipation during treatment with OXN (oxycodone+naloxone) compared with OXY (oxycodone) and placebo based on the patient bowel function index (difficulty of bowel movement, feeling of incomplete bowel evacuation, constipation self-assessment).

Two patients suffering from Parkinson's disease were among the subjects participating in the study.

### Overall Study Design and Plan:

This was a multicenter, randomised, double-blind, placebo- and active-controlled, double-dummy, parallel group study in men and women with low back pain (LBP) adequately controlled by an opioid analgesic. The maintenance of analgesia design was used to demonstrate the superiority of OXN over placebo on the time from the initial dose of study medication to multiple (ie, recurring) pain events (inadequate analgesia). 464 subjects were randomised in a 1:1:1 ratio to one of three treatment groups and 463 subjects received either OXN, OXY, or placebo for up to 12 weeks during the Double-blind Phase.

Two patients suffering from Parkinson's disease participated in the study; one of them was in the OxyPR group, whereas the other PD patient was in the OXN PR group.

This study was composed of three phases: a Pre-randomisation Phase, a Double-blind Phase, and an Extension Phase (The core study was the Pre-randomisation Phase and Double-blind Phase.). The Pre-randomisation Phase contained two periods: the Screening Period and the Run-in Period. The Screening Period involved prospective assessments and an opioid medication taper and was designed to qualify subjects for participation in the Run-in Period. The Run-in Period was designed to titrate OxyIR to analgesic effect, qualify subjects for participation in the Double-blind Phase, and enable identification of a dose equivalent for the study medication to be used after randomisation. The Double-blind Phase was designed to assess the safety and efficacy of OXN compared with placebo as a treatment for moderate to severe chronic nonmalignant pain. Available for those subjects who completed the Double-blind Phase, the Extension Phase was designed to assess the long-term safety of OXN for up to 12 additional months.

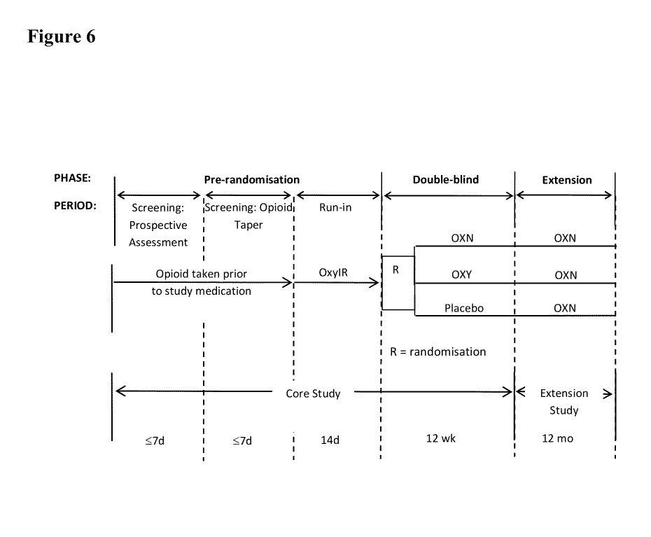

FIG. 6 shows the corresponding study diagram.

### Pre-Randomisation Phase:

the Pre-randomisation Phase duration was up to 28 days. The Pre-randomisation Phase, containing a Screening Period and Run-in Period, was designed to (a) assess inclusion/exclusion criteria, (b) confirm that opioids were required to treat the subject's moderate to severe LBP, (c) determine if the subject could achieve adequate analgesia with and tolerate immediate-release oxycodone, and (d) identify the dose of study medication used during the Double-blind Phase.

### Screening Period:

the Screening Period duration was up to 14 days. To be eligible to enter the Screening Period, subjects had to be at least 18 years of age and have a documented history of moderate to severe chronic pain of low back that required around-the-clock opioid therapy; the LBP had to be adequately managed by an opioid analgesic for at least the past 2 weeks.

### Prospective Assessment:

the Prospective Assessment duration was up to 7 days and involved signing of the informed consent as outlined above, enrolling the subject in the study, and reviewing eligibility for study enrollment. A subset of the inclusion/exclusion criteria could be verified at Visit 1. Subjects meeting all Screening inclusion/exclusion criteria (including all clinical laboratory test requirements) began the Opioid Taper at Visit 2.

### Opioid Taper:

the Opioid Taper duration was up to 7 days and involved down-titrating the subject's opioid medication until the subject demonstrated the need for continued opioid treatment, and reviewing eligibility for the Run-in Period. Down titration was performed according to the American Pain Society Opioid Tapering Algorithm. Open-label OxyIR was prescribed q4-6 h as needed (PRN) at a dose of ¼ the total daily opioid medication dose equivalent. Investigators instructed subjects to take a dose of OxyIR only when their Pain Intensity Scale Score (“Pain Right Now”) was ≧5.

After Visit 2, subjects completed diaries daily to record rescue medication (OxyIR) use, pain scores, and rate withdrawal symptoms. Withdrawal symptoms recorded on the SOWS were not recorded as adverse events unless they were of sufficient severity to be reported spontaneously by the subject. Members of the site study staff contacted subjects by telephone every 2 days. The staff asked subjects about their pain and OxyIR use. Investigators provided instructions to the subjects regarding any opioid medication dosing changes.

Subjects were asked to return to the study center 7 days after Visit 2/at the end of the Opioid Taper procedure, or as soon as possible after the Investigator preliminarily determined that the subject was appropriate for possible entry into the Run-in Period. To continue in the study and enter the Run-in Period, subjects had to 1) report unacceptable pain control for 2 consecutive days within 7 days after initiation of the opioid medication taper. A day of unacceptable pain control was defined as: Pain Intensity Scale (“Average Pain over 24 Hours”) score ≧5 or Pain Intensity Scale (“Pain Right Now”) score ≧5 accompanied by rescue medication dosing ≧2 times over one day) 2) Demonstrate no opioid withdrawal, defined as a Modified Subjective Opiate Withdrawal Scale (SOWS) score >24 or an increase of >15 points from Modified SOWS score assessed during the Prospective Assessment during the Screening Period (ie, baseline).

Subjects who did not demonstrate the need for opioid treatment within the first 6 days of the Opioid Taper or did not meet other inclusion/exclusion criteria did not continue in the study and resumed their prestudy treatment for pain, upon consultation with the Investigator. An early discontinuation CRF page was completed for subjects who did not enter the Run-in Period.

### Run-in Period:

The Run-in Period duration was 14 days. During the Run-in Period, the subjects' LBP was treated with OxyIR titrating the OxyIR to analgesic effect. Investigators converted subjects to an appropriate dose of OxyIR based on their effective opioid medication dose. OxyIR was dosed q4-6 h PRN and titrated according to the Investigator's judgment.

After Visit 3, subjects completed diaries daily to record OxyIR use, pain scores, and bowel function. Members of the site study staff contacted subjects by telephone every 2 days. The staff asked subjects about their pain and OxyIR use. Investigators provided instructions to the eligible subjects regarding any OxyIR dosing changes.

To continue in the study and be randomised, subjects had to 1) tolerate OxyIR treatment during the Run-in Period; 2) report an average Pain Intensity Scale Score (“Average Pain over 24 Hours”; (0-10)) of <4.5 taking the mean value over the last 7 days of the Run-in Period with 15 to 45 mg/d OxyIR; 3) have appropriate and legible diary completion.

At Visit 4, site study staff reviewed eligibility for randomisation and then randomised appropriate subjects into the Double-blind Phase. Randomisation was done in blocks by country and centralised with the interactive voice response system (IVRS).

Subjects who did not experience satisfactory pain relief for their LBP with OxyIR or did not meet other inclusion/exclusion criteria were not randomised into the study and resumed their prestudy treatment for breakthrough pain, upon consultation with the Investigator.

### Double-Blind Phase:

the Double-blind Phase duration was 12 weeks. In the Double-blind Phase, subjects' LBP was treated with double-blind study medication (ie, OXN, OXY, or placebo). Subjects were randomised in a 1:1:1 ratio to OXN, OXY, or placebo. Investigators provided instructions to the subjects regarding study medication and laxative dosing. Subjects were converted from the effective dose of OxyIR established during the Run-in Period to the equivalent dose level of the double-blind study medication. Study medication dosing was q12 h with a fixed and symmetrical dose. Open-label OxyIR was provided as add-on therapy (ie, rescue medication). OxyIR was prescribed q4-6 h PRN at a dose of ¼ the total daily study medication dose. Investigators instructed subjects to take a dose of OxyIR only when their Pain Intensity Scale Score (“Pain Right Now”) was ≧5. Subjects discontinued use of laxatives for the first 3 days after randomisation. After post-randomisation Day 3, subjects could take a laxative(s) dosed at the discretion of the Investigator.

After Visit 4, subjects completed diaries daily to record rescue medication (OxyIR) use, pain scores, and bowel function. Subjects were instructed to contact the site by telephone to report any adverse events.

Subjects received double-blind study medication for approximately 12 weeks. Study visits occurred at Weeks 2, 4, 8, and 12.

Subjects who did not tolerate the study medication or developed signs or symptoms that contraindicated continuation of opioid therapy were discontinued from the study. Site study staff members discontinued the subject from the study and the subject returned to the clinic for appropriate therapy according to standard of care.

For subjects who prematurely stopped participation in the study, Investigators questioned subjects about their primary reason for discontinuing from the study, which was recorded in the CRF. Site study staff members followed subjects after the last dose of study medication for 7 days to collect non-serious adverse events, for 30 days to collect serious adverse events and obtain non-serious adverse event outcome information, and for serious adverse events, until the event resolved, or the event or sequelae stabilised.

For subjects who discontinued their study medication, either after completion or discontinuation from the Double-blind Phase, site study staff contacted those subjects by telephone eight days after discontinuing their study medication. Site staff asked subjects about their symptoms and current analgesic treatment. All responses were recorded in the CRF.

### Selection of Study Population:

subjects had moderate to severe chronic LBP, which served as a model for non-malignant pain. 464 subjects were randomised into the Double-blind Phase. 676 subjects were screened in the Pre-randomisation Phase to achieve this sample size.

### Inclusion Criteria:

subjects had to meet all the following criteria to be included in this study:
Males and females at least 18 years of age (Females less than one year post-menopausal had to have a negative serum or urine pregnancy test recorded within 72 hours prior to the first dose of study medication, be non-lactating, and willing to use adequate and reliable contraception throughout the study.).
Documented history of moderate to severe chronic pain of low back that required around-the-clock opioid therapy.
Nonmalignant low back pain adequately managed by an opioid analgesic for at least the past 2 weeks.
Subjects who required continuation of daily opioid analgesic treatment and were likely to benefit from chronic opioid therapy for the duration of the study.
Subjects willing and able to participate in all aspects of the study, including use of oral medication, completion of subjective evaluations, attending scheduled clinic visits, completing telephone contacts, and compliance with protocol requirements as evidenced by providing written, informed consent.

### Exclusion Criteria:

subjects who met any of the following criteria were excluded from this study:
Any history of hypersensitivity to oxycodone, naloxone, or related products.
Subjects currently taking the equivalent of <10 mg or >40 mg/d oxycodone.
Subjects diagnosed with cancer, not including basal cell carcinoma.
Active alcohol or drug abuse with severity sufficient to place the subjects at risk.
Evidence of clinically significant cardiovascular, renal, hepatic, gastrointestinal (paralytic ileus), or psychiatric disease, as determined by medical history, clinical laboratory tests, Electrocardiogram (ECG) results, and physical examination, that would have placed the subject at risk upon exposure to the study medication or that could confound the analysis and/or interpretation of the study results.
Abnormal aspartate aminotransferase (AST; SGOT), alanine aminotransferase (ALT; SGPT), or alkaline phosphatase levels (>3 times the upper limit of normal) or an abnormal total bilirubin and/or creatinine level(s) (outside of the reference range).
Surgery completed 2 months prior to the start of the Screening Period, planned surgery during the 12-week Double-blind Phase, or any other pharmacological or non-pharmacological intervention that would have influenced pain during the study (not including chemotherapy) or precluded completion of the study.
Subjects taking, or who had taken, naloxone or an experimental drug 30 days prior to the start of the Screening Period.
Subjects with a history of 2 or greater low back surgeries.

### Run-in Period Entrance Criteria:

these criteria were assessed at the end of the Opioid Taper. Subjects had to meet the following criteria to enter the Run-in Period:
Report unacceptable pain control for 2 consecutive days within 7 days after initiation of the opioid medication taper. A day of unacceptable pain control was defined as: 1) Pain Intensity Scale (“Average Pain over 24 Hours”) score ≧5 or 2) Pain Intensity Scale (“Pain Right Now”) score ≧5 accompanied by rescue medication dosing ≧2 times over one day.
Demonstrate no opioid withdrawal defined as a Modified Subjective Opiate Withdrawal Scale (SOWS) score >24 or an increase of >15 points from the Modified SOWS score assessed during the Prospective Assessment during the Screening Period (ie, baseline).

### Randomisation Criteria:

these criteria were assessed at the end of the Run-in Period. Subjects had to meet the following criteria to be randomised:
Subjects who tolerated OxyIR treatment during the Run-in Period.
Subjects who reported an average Pain Intensity Scale Score (“Average Pain over 24 Hours”; (0-10)) of <4.5 taking the mean value over the last 7 days of the Run-in Period with 15-45 mg/d OxyIR.
Subjects with appropriate and legible diary completion.

### Schedule of Visits and Procedures:

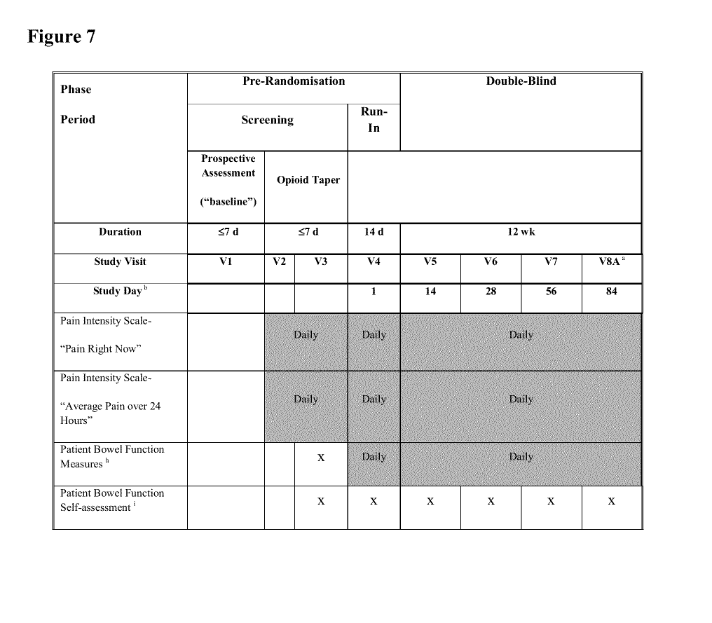

FIG. 7 shows the schedule of visits and procedures/CRF modules for the Core Study.

### Efficacy Assessments:

### Pain:

The primary efficacy variable was the time from the initial dose of study medication to recurring pain events during the Double-blind Phase. A pain event was demonstrated by unacceptable pain control for 2 consecutive days. Each pain event was 2 discrete days, eg, there could be a maximum of 2 pain events in 4 days. A day of unacceptable pain control was defined as:

1) Pain Intensity Scale (“Average Pain over 24 Hours”) score ≧5 or
2) Pain Intensity Scale (“Pain Right Now”) score ≧5 accompanied by rescue medication dosing ≧2 times over one day.
OR subjects could have a pain event by:
3) Study discontinuation due to lack of therapeutic effect.

The pain event criteria were composed of the following variables:
Pain Intensity Scale: The Pain Intensity Scale assessed subjects' pain on a 10-point ordinal scale (0=No pain, 10=Pain as bad as you can imagine). Subjects did retrospectively assess their average pain over the past 24 hours each evening (“Average Pain over 24 hours”), and assessed their pain at the time immediately prior to rescue medication dosing (“Pain Right Now”). Subjects recorded their pain scores in their paper diaries.
Rescue medication intake (dose, time). Subjects recorded their dosing information in their paper diaries.
Reason for discontinuation from the Double-blind Phase. Investigators interviewed the subjects to determine the subject's single primary reason for discontinuation. The Investigator recorded the appropriate discontinuation category (eg, “lack of therapeutic effect”) in the CRF and filled in the AE CRF or Serious Adverse Event (SAE) Data Form, if applicable.

### Pain Intensity Scale:

The Pain Intensity Scale assessed subjects' pain on an 10-point ordinal scale (0=No pain, 10=Pain as bad as you can imagine). Subjects retrospectively assessed their average pain over the past 24 hours each evening (“Average Pain over 24 hours”). Subjects recorded their pain scores in their paper diaries.

A subject's BFI score was the arithmetic mean of the following items (assessed at each visit):
1) difficulty of bowel movement (over the last 7 days) (0-10; 0=easy/no difficulty, 10=severe difficulty); 2) feeling of incomplete bowel evacuation (over the last 7 days) (0-10; 0=not at all, 10=very strong); 3) judgment of constipation (over the last 7 days) (0-10; 0=not at all, 10=very strong).

### Treatments Administered:

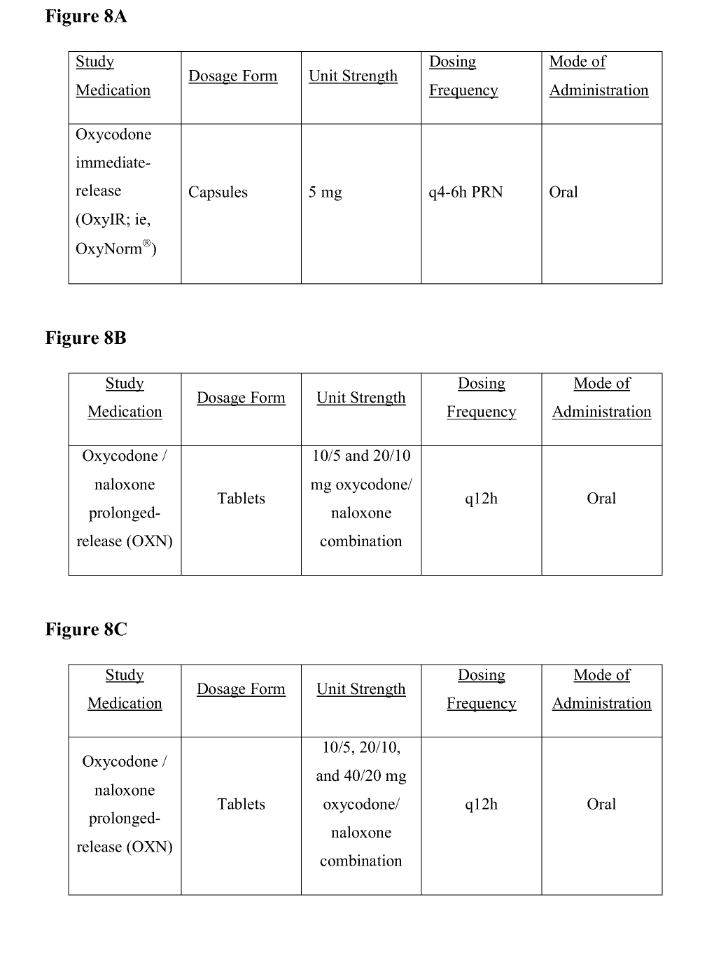

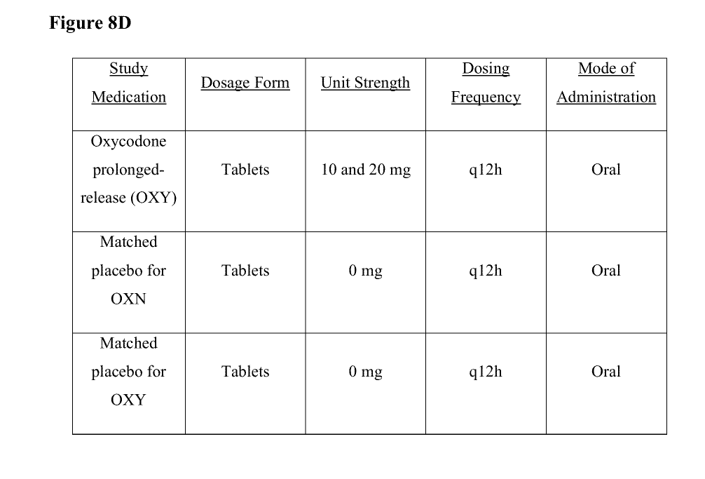

OxyIR Use During Open-Label Treatment—Opioid Taper, Run-in Period, Double-Blind Phase, and Extension Phase (See FIG. 8A)

During the Screening Period Opioid Taper, subjects could receive OxyIR q4-6 h PRN as rescue medication at a dose of ¼ their total daily opioid medication dose. Subjects were instructed to take a dose of OxyIR only when their Pain Intensity Scale (“Pain Right Now”) Score was ≧5.

At the time of the demonstration of the need for continued opioid treatment during the Screening Period Opioid Taper, subjects entering the Run-in Period discontinued their opioid medication (if not already discontinued) and converted to an appropriate dose of OxyIR. During the Run-in Period, the OxyIR dose was titrated to effect. The target dose of OxyIR was 20 or 40 mg/d. At the start of the Double-blind Phase, all randomised subjects were converted from OxyIR to an equivalent study medication dose. During the Double-blind Phase, all subjects could receive OxyIR q4-6 h PRN as rescue medication at a dose of ¼ the total daily study medication dose. Subjects were instructed to take a dose of OxyIR only when their Pain Intensity Scale Score was ≧5. OxyIR was also provided to subjects for the first 7 days of the Extension Phase.

### Double-Blind Treatment—Double-Blind Phase (FIG. 8B)

During the Double-blind Phase, subjects randomised to the OXN treatment group received blinded OXN and matched OXY placebo. Dosing was fixed and symmetrical at the equivalent of the effective OxyIR dose identified during the Run-in Period.

### Open-Label Treatment—Extension Phase (FIG. 8C)

During the Extension Phase, subjects who completed the Double-blind Phase and elected to enter the Extension Phase received open-label OXN. Subjects entering the Extension Phase switched to 20/10 mg/d oxycodone/naloxone. Dose titration was permitted at the discretion of the Investigator.

### Reference Treatment:

Double-Blind Treatment—Double-Blind Phase (FIG. 8D)

During the Double-blind Phase, subjects randomised to the OXY treatment group received blinded OXY and matched OXN placebo. Dosing was fixed and symmetrical at the equivalent of the effective OxyIR dose identified during the Run-in Period.

During the Double-blind Phase, subjects randomised to the placebo group received blinded OXY placebo and OXN placebo. Dosing was fixed and symmetrical at the equivalent of the effective OxyIR dose identified during the Run-in Period.

Subjects took the first dose of double-blind study medication at home in the evening.

### Method of Administration:

the blinded study medication (ie, OXN, OXY, or placebo) was administered orally, prescribed q12 h. The open-label rescue medication (ie, OxyIR) was administered orally, prescribed q4-6 hours. Subjects were instructed to only take a dose of rescue mediation if their pain intensity “Pain Right Now” score was at least 5.

### Blinding:

the study medication (OXN, OXY, placebos) was packaged in a double-blind, double-dummy manner, rendering the active tablets indistinguishable from the matched placebo tablets.

During the Double-Blind Phase, the subject and all personnel involved with the conduct and the interpretation of the study, including the Investigators, investigational site personnel, and the Sponsor's and CRO's staff, were blinded to the medication codes. Randomisation data were kept strictly confidential, filed securely by the Sponsor, and accessible only to authorised persons per Sponsor's Standard Operating Procedures (SOPs) until the time of unblinding.

### Subject Disposition:

The sites enrolled 751 subjects into the study. 676 subjects entered the Opioid Taper. Of these, 73 subjects discontinued during the Opioid Taper. The primary reason for discontinuation in the Opioid Taper was the experience of adverse events (24 subjects, 3.6%). 139 subjects discontinued during the Run-in (Titration) Period. The primary reason for discontinuation in the Run-in Period was lack of therapeutic effect (68 subjects, 11.3%). 464 subjects were randomised into the study. Table 5 summarises the disposition of 463 subjects randomised to treatment in the Double-blind Phase by treatment group (excluding 1 subject, who was excluded from the full analysis as he did not receive study medication after randomisation).

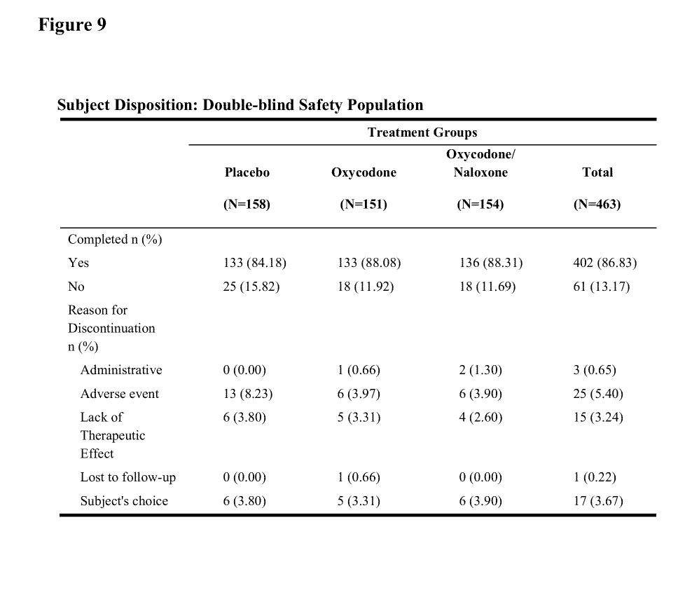

FIG. 9 shows the subject Disposition in the double-blind safety population. Adverse events were the major reason for premature termination (5.4%). The overall percentage of subjects who discontinued was higher among subjects who received placebo (15.8%) than among subjects who received oxycodone (11.9%) or oxycodone/naloxone (11.7%).

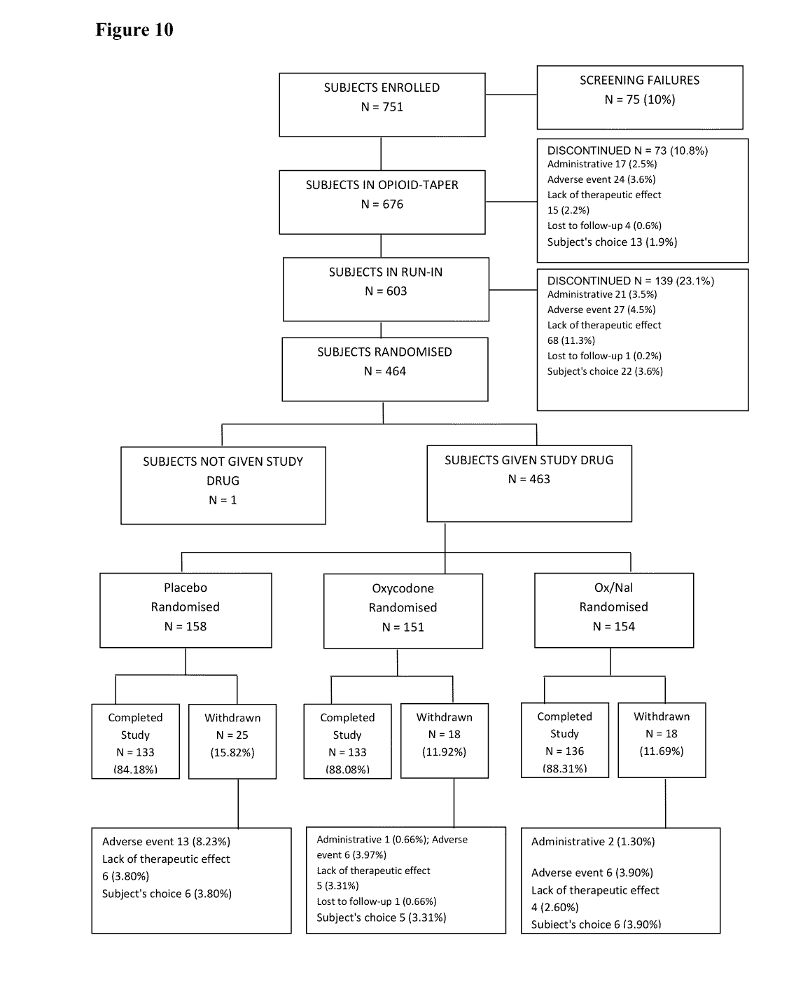

FIG. 10 displays the disposition of subjects in study II.

### Results for the Two PD-Patients:

The BFI and Pain Intensity scores were determined at the visits as described above. One PD patient (subject “D”) received OXN for treatment, whereas the other PD patient (subject “E”) received OXY.

| Subject | Visit Number | BFI | Pain | Treatment |
|---|---|---|---|---|
| D | 1 | 67.00 | 5.00 | OXN |
| D | 2-3 | 50.00 | 4.75 | OXN |
| D | 3-4 | 23.00 | 4.78 | OXN |
| D | 4-5 | 3.00 | 3.64 | OXN |
| D | 5-7 | 23.00 | 4.50 | OXN |
| D | 7-8 | 53.00 | 4.39 | OXN |
| E | 1 | 27.00 | 5.00 | OXY |
| E | 2-3 | 3.00 | 3.00 | OXY |
| E | 3-4 | 3.00 | 3.00 | OXY |
| E | 4-5 | 7.00 | 3.00 | OXY |
| E | 5-7 | 17.00 | 3.00 | OXY |
| E | 7-8 | 10.00 | 3.00 | OXY |

### BFI-Score:

arithmetic mean of the following items:

1) Ease of defecation (numerical analogue scale [NAS], 0=easy/no difficulty; 100=severe difficulty); 2) Feeling of incomplete bowel evacuation (NAS, 0=not at all, 100=very strong); 3) Personal judgment of constipation (NAS, 0=not at all, 100=very strong). Each of the questions referred to the last 7 days for the subject.

### Pain-Score:

Average Pain on a 10-point ordinal scale, 0=no pain; 10=pain as bad as you can imagine.

### Analysis of the Data of Examples 1 and 2:

### Summary for BFI:

The data gained for the PD patient group receiving OXN (n=2; subjects A and D) and the data gained for the PD patient group receiving OXY (n=3; subjects B, C and E) can be displayed with mean values as follows:

| Treat- | | BFI: | | | | | | | |
|---|---|---|---|---|---|---|---|---|---|
| ment | | V1 | V2 | V3 | V4 | V5 | V6 | V7 | V8 |
| OXN | n = 2 | 60.1 | 51.6 | 29.8 | 13.1 | 19.8 | 14.8 | 31.5 | 28.1 |
| OXY | n = 3 | 58.4 | 47.1 | 49.9 | 43.4 | 46.8 | 56.8 | 54.4 | 56.7 |

Clearly, the treatment with OXN results in an increased bowel function compared to the treatment with OXY only.

### Summary for Pain:

The data gained for the PD patient group receiving OXN (n=2; subjects A and D) and the data gained for the PD patient group receiving OXY (n=2; subjects C and E) can be displayed with mean values as follows [patient B was excluded due to the lack of pain intensity scores at visits 5 to 8]:

| Treatment | | Pain: V1 | V2 | V3 | V4 | V5 | V6 | V7 | V8 |
|---|---|---|---|---|---|---|---|---|---|
| OXN | n = 2 | 4 | 3.9 | 3.9 | 3.9 | 3.3 | 3.7 | 3.2 | 3.7 |
| OXY | n = 2 | 4.5 | 3 | 3.5 | 3 | 4 | 3.5 | 3.5 | 3.5 |

Thus, the treatment with OXN results in an as efficient pain treatment as pain treatment with OXY only.

### Example 3

### Improvement of Pain and LID in PD Patients

The following data are based on case studies wherein PD patients were stationary treated with OXN PR (oxycodone+naloxone in a prolonged release dosage form).

The following table lists the age, the sex, the duration of PD, the indication and the amount of oxycodone in the dosage form (in mg) administered. Naloxone was present in each of the dosage forms in 0.5× the amount of oxycodone. Furthermore, the table provides information on the effect of OXN PR on pain and LID as well as adverse events.

| | | | | | | | Adverse |
|---|---|---|---|---|---|---|---|
| Patient | Age | Sex | Duration | Indication | OXN | Effect | events |
| 1 | 69 | F | 16 | arthritis + LID | 10 mg | Pain: ++, | none |
| | | | | | | LID: + | |
| 2 | 73 | M | 23 | LBP + LID | 2 × 15 mg | Pain: ++, | none |
| | | | | | | LID: +/− | |
| 3 | 68 | M | 11 | Lumb.Disc. + LID | 3 × 20 mg | Pain: ++, | none |
| | | | | | | LID: + | |
| 4 | 72 | M | 7 | LBP, hip | 20 mg | Pain: ++ | none |
| 5 | 81 | F | 2 | LBP, Gonarth. | 2 × 5 mg | pain: − | sleep apnea |
| 6 | 74 | F | 5 | Osteoporosis | 2 × 20 mg | pain: + | constip.idem |
| 7 | 66 | F | 11 | LBP, | 3 × 10 mg | pain: +, | none |
| | | | | Epic.uln. + LID | | LID: + | |
| 8 | 69 | M | 2 | LBP, | 2 × 10 mg | pain: ++ | none |
| | | | | lumb.fracture | | | |

The following abbreviations are used in the above table:
F: female/M: male;
LID: L-dopa induced dyskinesia;
LBP: lower back pain;
Yx indicates that the OXN dosage form was administered Y times within a 24 h interval (2 × 15 mg = 2 times 15 mg OXN within 24 h)
+ indicates an improvement/
++ indicates a strong improvement of the condition;
+/− indicates no change in the condition;/
− indicates a worsening of the condition.

For patient 1, a more detailed case report as follows was recorded:
Patient: 69 year old female patient with PD of 16 years duration with fluctuations and dyskinesias and severe pain in her right foot after arthritis and fracture; no cognitive decline; rheumatoid arthritis known and treated with methotrexat for years;
Conditions: Severe motor symptoms with H+ Y stage 4, dyskinesias. UPDRS III (motor part): 19 on admission, no change on motor symptoms (19) but reduction of troublesome dyskinesias after therapy with OXN 10 mg during daytime and improvement of hypokinesia. No side effects by OXN, no constipation reported, but Macrogol (13 mg) continued.

### Example 4

### Clinical Study Protocol for Assessing the Efficacy of OXN PR in Parkinson's Disease (PD) Patients: a Randomised Placebo Controlled Study of OXN PR for Severe PD Associated Pain

### Objectives:

To demonstrate superiority of OXN PR compared to placebo with respect to analgesic efficacy in subjects with chronic severe pain associated with PD, as assessed by averaged 24 hour pain scores collected for 7 days prior to the clinic visits; to demonstrate improvement in the subject's condition, relative to baseline, as measured on the Clinical Global Impression-Improvement scale (CGI-I) and separately the Patient Impression-Improvement scale (PGI-I); to assess the effect of OXN PR on motor symptoms of PD; to assess the effect of OXN PR on non-motor symptoms; to assess the effect of OXN PR on dyskinesia; to assess the effect of OXN PR on sleep; to assess the effect of OXN PR on Quality of Life; to assess the tolerability of OXN; to assess the frequency of rescue medication intake.

### Study Design:

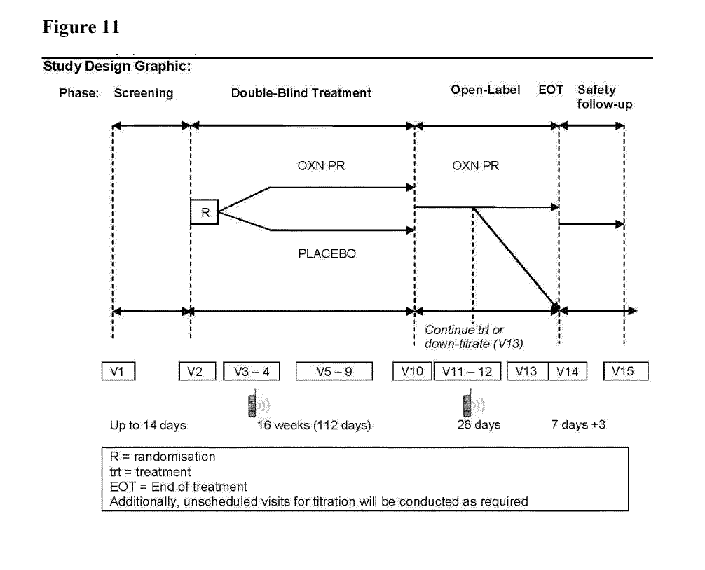

a multicentre, double-blind, randomised, placebo controlled, parallel-group study in male and female subjects to assess the efficacy and tolerability of OXN PR to control PD's related chronic severe pain. An overview of the study scheme can be found in FIG. 11.

### Screening:

Subjects will undergo screening which may take 7 (minimum) to 14 days.

### Randomisation:

Subjects who have consented to participate and who are eligible for treatment will be randomised to receive either OXN PR or matched placebo.

### Double-Blind Phase:

Subjects will be followed up by telephone in the first week and attend visits at week 1, 2 (+/−3 days), 4, 8, 12 and 16 (+/−5 days). All subjects will be started on OXN5/2.5 mg PR twice daily (OXN 10/5 mg PR total daily dose) and may be titrated to a maximum daily dose of OXN20/10 twice daily (OXN40/20 mg PR total daily dose) or matched placebo.

### Open-Label Phase:

Subjects may enter an Open-Label Phase of up to 4 weeks duration following completion or who discontinue early but who have had at least 8 weeks study treatment.

### Safety Follow-Up:

Subjects will be followed up for safety 7-10 days after receiving the last dose of study treatment. Note: Subjects may be prescribed OXN PR from the end of study participation (Visit 10 or Visit 14).

### Rescue Medication:

Rescue medication in the Double-Blind Phase will be levodopa and benserazide hydrochloride combination. Rescue medication in the Open-Label Phase will be oxycodone immediate release (OxyIR).

### Selection of Study Population:

Subjects will have idiopathic PD and be suffering from severe PD associated pain. Approximately 210 subjects will be randomised into the Double-Blind Phase to achieve 172 subjects with an assessment at 16 weeks for the primary efficacy variable. An adequate number of subjects (estimated at 250) will be screened to achieve this sample size.

### Inclusion Criteria:

1: Males and females, age of 25 years or over (the Double-Blind Phase rescue medication is not licensed for use in under 25 year olds); 2: able to provide written informed consent; 3: primary diagnosis of PD diagnosed by an expert as determined by the UK Parkinson's Disease Society Brain Bank Clinical Diagnostic Criteria (1992); 4: Parkinson's disease Stage II-IV (Hoehn & Yahr staging system); 5: severe pain graded in at least 1 of the sub sections of the Chaudhuri and Schapira (2009) pain classification system; 6: an average pain score of 6 or above on an 11 point NRS, over the previous 7 days determined using diary scores of averaged 24 hour pain in the 7 days leading up to Randomisation (assessed at Visit 2); 7: female subjects less than one year post-menopausal must have a negative serum or urine pregnancy test recorded prior to the first dose of study medication, be non-lactating, and willing to use an adequate and highly effective method of contraception throughout the study; 8: subjects who, based on the Investigators' judgement, are likely to benefit from WHO step III opioid therapy for the duration of the study; 9: subjects must not have received opioid containing medication in the last 6 months on a regular basis (i.e. prescribed medication or more than occasional self medication use for cough, cold etc.); 10: receiving stable treatment for PD for at least 4 weeks prior to randomisation, the dose of which is expected to remain consistent throughout the Double Blind Phase; 11: in the Investigator's opinion, the subject does not have visual or auditory impairments that would reduce their ability to complete study questionnaires or be unable to receive instructions for these; 12: concomitant medication (including co-analgesic) use anticipated to remain stable throughout the Double-Blind Phase of the study; 13: subjects willing and able to participate in all aspects of the study and comply with the use of study medication.

### Open-Label Extension Inclusion Criteria:

Subjects must still meet general inclusion criteria for Double-Blind Phase; subjects do not have to meet inclusion 5, 6, 9 & 12; subjects must have completed the Double-Blind Phase or discontinued early but have had at least 8 weeks treatment with study medication.

### Exclusion Criteria:

Cognitive impairment as assessed with the MMSE scoring 24 or less; history of psychosis (hallucinations, delusions, etc.); history of drug or alcohol abuse or current compulsive addictive use of drugs or alcohol; Parkinsonian-like disease secondary to drug therapy side-effects e.g. due to exposure to medications that deplete dopamine (reserpine, tetrabenazine) or block dopamine receptors (neuroleptics, antiemetics); Parkinson-plus syndromes e.g. progressive supranuclear palsy (PSP) and the multiple system atrophies (MSA); females who are pregnant or lactating; any other contraindications to use of the opioid study medication(s) as per the SmPC/IB; any other contraindications to use of the study Double-Blind Phase rescue medication as per the SmPC; Subjects with any of the following as determined by medical history, clinical laboratory tests, ECG results, and physical examination, that would place the subject at risk upon exposure to the study medication: myxoedema/untreated hypothyroidism/Addison's disease/increase of intracranial pressure/uncontrolled seizures or convulsive disorder/evidence of clinically significant cardiovascular, renal, hepatic, gastrointestinal (e.g. paralytic ileus), or psychiatric disease (subjects with controlled co-morbidities may be included following agreement with the Medical Monitor).

### Contraindicated Treatments:

treatment with Deep Brain Stimulation; subjects receiving hypnotics or other central nervous system (CNS) depressants that, in the Investigator's opinion, may pose a risk of additional CNS depression with opioids study medication; subjects presently taking, or who have taken, naloxone or naltrexone ˜30 days prior to the Screening Visit; subjects who have received an investigational medicinal product within 30 days of study entry (defined as the start of the Screening Phase); any current use of an opioid other than the study medication provided; subjects with a positive urine drug test at Screening Visit 1, which indicates unreported illicit drug use or unreported use of a concomitant medication not required to treat the Subjects' medical condition(s).

### Test Treatment, Dose and Mode of Administration:

The following doses will be allowed for twice daily use in accordance with the SmPC: Oxycodone/naloxone prolonged-release (OXN PR) in the form of tablets; unit strengths: OXN5/2.5 mg PR/OXN10/5 mg PR/OXN15/7.5 mg PR/OXN20/10 mg PR; dosing frequency: q12 h; oral administration. All subjects will be treated for up to a maximum of 16 weeks (+/−5 days) prior to the open label phase. Subjects will start the double-blind phase on a dose of OXN5/2.5 mg PR or matching placebo twice daily. Titration up to the maximum daily dose of OXN40/20 mg PR (e.g. OXN20/10 mg PR twice daily) is permitted.

### Reference Treatment, Dose and Mode of Administration:

The study will have matching placebos for OXN PR; dosing frequency: q12 h; oral administration.

### Concomitant Medication Including Rescue:

PD: Subjects should ideally remain on a stable dose of medicines given for PD throughout the study. Any required changes in PD treatment must be recorded along with any changes in disease symptoms. Laxative Medication Subjects who use laxatives prior to study start should ideally continue as per the pre-study dosing regimen. Any change in dose must be recorded. Rescue Medication in the double-blind phase: Levodopa and benserazide HCl combination in the form of tablets; unit strength: 100/25 mg (max 3 tablets daily); dosing frequency: PRN; oral administration. Rescue Medication in open-label phase: Oxycodone immediate release (OxyRI) in the form of capsules; unit strength: 5 mg (max daily: 30 mg); dosing frequency: PRN; oral administration.

### Treatment Schedule:

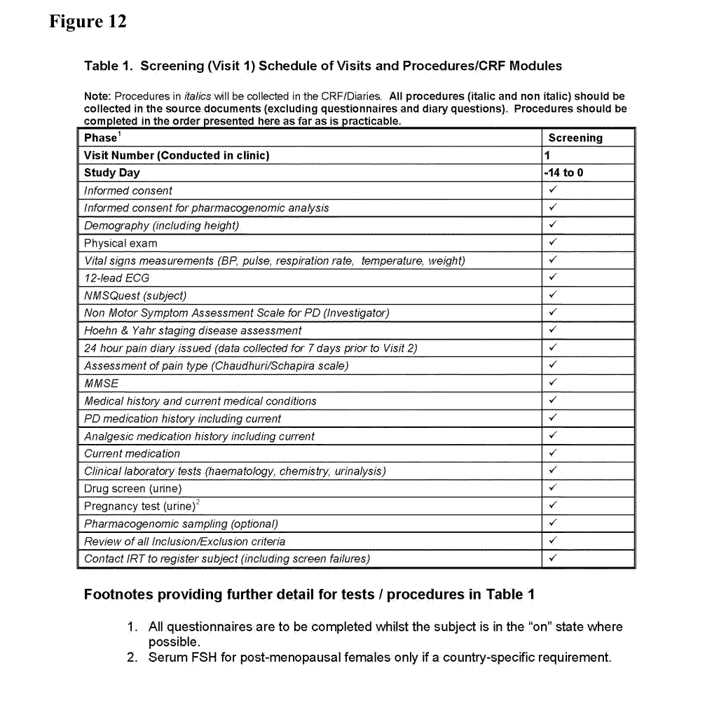

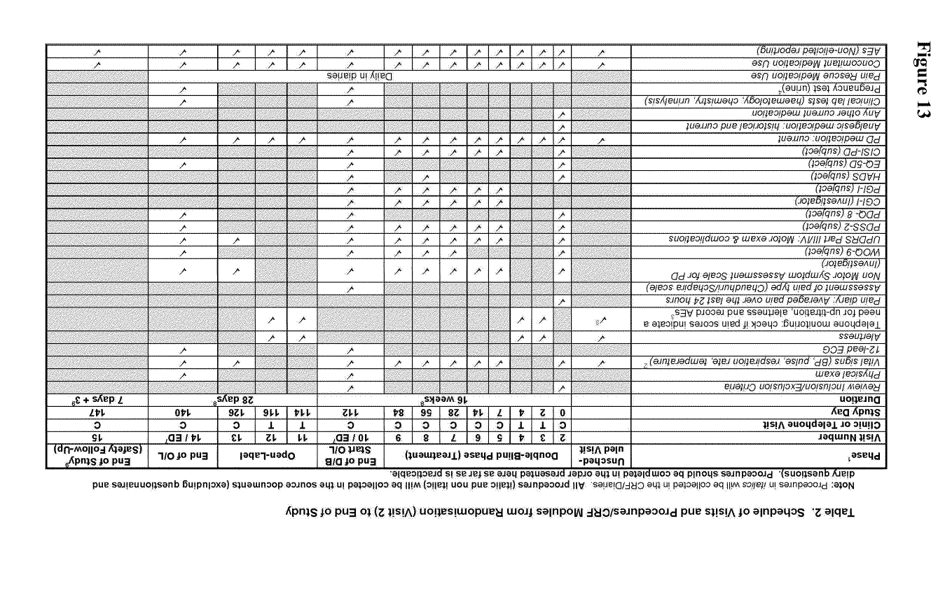

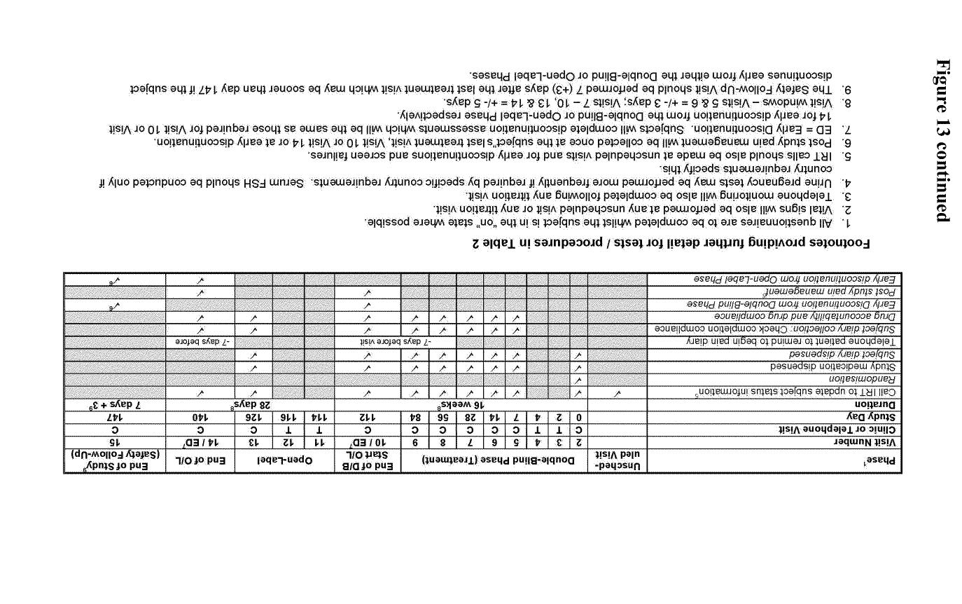

In the screening phase, subjects will undergo tests and procedures, and complete interviews and questionnaires in accordance with FIG. 12 (Table 1). In the randomisation phase, subjects will undergo tests and procedures, and complete interviews and questionnaires in accordance with FIG. 13 (Table 2). Randomisation will be completed once all inclusion and exclusion criteria are verified. Subjects who qualify for entry into the Double-Blind Phase of the study will be randomised to OXN PR or OXN PR matching placebo in a 1:1 ratio. The IRT will be contacted to update subject information and allocate medication packs to be dispensed. At the start of the Double-Blind Phase subjects will start with OXN5/2.5 mg PR or matching placebo twice daily. A subject diary will be dispensed for recording of all rescue medication use and to record average 24 hour pain scores. In the double-blind phase and the open-label phase (Visit 10), subjects will undergo tests and procedures, and complete interviews and questionnaires in accordance with FIG. 13 (Table 2). The safety follow-up (Visit 15), will take the form of a telephone call or clinic visit 7 days (+3) after the last dose of study medication. The purpose of the visit is to assess safety including follow up of any ongoing AEs (AE FU) and to record any new AEs that may have occurred and check for any changes in concomitant medications. This visit should also be completed for any subject that discontinues early from the study.

### Efficacy Assessments:

The primary endpoint for the primary comparison of OXN PR vs. placebo: Averaged 24 hour pain scores collected for 7 days preceding the study clinic visit (week 16). The following key secondary endpoints for the primary comparison of OXN PR vs. placebo will be tested in a hierarchical testing strategy: Averaged 24 hour pain scores collected for the 7 days preceding individual clinic visits during the Double-Blind Phase; CGI-I: Percentage of responders (defined as a response of “Much improved” or “Very much improved”) on the CGI-I scale (as defined by the Investigator). Other exploratory endpoints: Percentage of responders (defined as a response of “Much improved” or “Very much improved”) on the PGI-I scale (as defined by the subject); Change from baseline in the total score and domains of the Non Motor Symptom Assessment Scale for Parkinson's Disease to the end of the Double-Blind Phase (week 16); Change from baseline in the total score of the UPDRS Part III/IV Motor Examination to the end of the Double-Blind Phase (week 16); Change from baseline in percentage of subjects meeting wearing off criteria (defined as the presence of at least one symptom in the WOQ-9 with improvement after the next dose of anti-Parkinsonian medication); Change from baseline in the total score of the CISI-PD to the end of the Double-Blind Phase (week 16); Frequency of rescue medication use during the Double-Blind Phase; Change from baseline in the total score of the PDSS-2 to the end of the Double-Blind Phase (week 16); Change from baseline in the total score of the PDQ-8 to the end of the Double-Blind Phase (week 16); Change from baseline in EQ-5D index score to the end of the Double-Blind Phase (week 16); Change from baseline in the anxiety domain score of the HADS to the end of the Double Blind Phase (week 16); Change from baseline in the depression domain score of the HADS to the end of the double-blind phase.

Further preferred embodiments of the present invention relate to:
1. A pharmaceutical dosage form comprising an opioid agonist and an opioid antagonist for use in the treatment of Parkinson's disease and/or at least one symptom thereof
2. Dosage form according to 1, wherein the opioid agonist is selected from the group comprising morphine, oxycodone, hydromorphone, dihydroetorphine, etorphine, nalbuphine, propoxyphene, nicomorphine, dihydrocodeine, diamorphine, papavereturn, codeine, ethylmorphine, phenylpiperidine, methadone, dextropropoxyphene, buprenorphine, pentazocin, tilidine, tramadol, tapentadol, hydrocodone and pharmaceutically acceptable salts thereof; and wherein the opioid antagonist is selected from the group comprising naltrexone, naloxone, nalmefene, nalorphine, nalbuphine, naloxonazine, methylnaltrexone, ketylcyclazocine, norbinaltorphimine, naltrindole and pharmaceutically acceptable salts thereof
3. Dosage form according to 2, wherein the opioid agonist is oxycodone or a pharmaceutically acceptable salt thereof and the opioid antagonist is naloxone or a pharmaceutically acceptable salt thereof
4. Dosage form according to 3, wherein the dosage form comprises oxycodone or a pharmaceutically acceptable salt thereof in an amount range of equivalent to 1 mg to 160 mg oxycodone HCl and naloxone or a pharmaceutically acceptable salt thereof in an amount range of equivalent to 0.5 mg to 80 mg naloxone HCl.
5. Dosage form according to 3 or 4, wherein the dosage form comprises oxycodone or a pharmaceutically acceptable salt thereof and naloxone or a pharmaceutically acceptable salt thereof in a 2:1 ratio by weight.
6. Dosage form according to 2, wherein the opioid agonist is hydromorphone or a pharmaceutically acceptable salt thereof and the opioid antagonist is naloxone or a pharmaceutically acceptable salt thereof
7. Dosage form according to 6, wherein the dosage form comprises hydromorphone or a pharmaceutically acceptable salt thereof in an amount range of equivalent to 1 mg to 64 mg hydromorphone HCl and naloxone or a pharmaceutically acceptable salt thereof in an amount range of equivalent to 0.5 mg to 256 mg naloxone HCl.
8. Dosage form according to 6 or 7, wherein the dosage form comprises hydromorphone or a pharmaceutically acceptable salt thereof and naloxone or a pharmaceutically acceptable salt thereof in a 2:1, 1:1, 1:2 or 1:3 ratio by weight.
9. Dosage form according to any of 1 to 8, wherein the dosage form is a prolonged release dosage form.
10. Dosage form according to 9, wherein the dosage form comprises a prolonged release matrix.
11. Dosage form according to 10, wherein the matrix comprises a fatty alcohol and a hydrophobic polymer, preferably an alkylcellulose and more preferably ethylcellulose.
12. Dosage form according to any of 1 to 8, wherein the dosage form is an immediate release dosage form.
13. Dosage form according to any of 1 to 12, wherein the dosage form is an oral dosage form, preferably selected from the group comprising a tablet, a capsule, a multi-particulate, a dragée, a granulate and a powder.
14. Dosage form according to any of 1 to 13, wherein the at least one symptom of Parkinson's Disease is selected from a motor symptom including dyskinesia, hypokinesia, rigor and tremor; and a nonmotor symptom (NMS) including constipation; disturbed bowel function; urgency; nocturnia; cardiovascular symptoms; sleeping disorders; fatigue; apathy; drooling of saliva; difficulties in maintaining concentration; skin disorders; psychiatric disorders including depression and anxiety; respiratory symptoms; cough; dyspnea and pain.
15. Dosage form according to any of 1 to 14, wherein the dosage form is for use in the treatment of at least one symptom of Parkinson's Disease selected from dyskinesia, pain and constipation.
16. Dosage form according to 14 or 15, wherein the dyskinesia is an L-Dopa induced dyskinesia (LID).
17. Use of an opioid agonist in combination with an opioid antagonist in pharmaceutical dosage form for the treatment of Parkinson's disease and/or at least one symptom thereof.

1. A method for treating Parkinson's disease or at least one symptom of a patient suffering from Parkinson's disease comprising administering to a patient in need thereof a pharmaceutical dosage form comprising:
an opioid agonist or a pharmaceutically acceptable salt thereof; and
an opioid antagonist or a pharmaceutically acceptable salt thereof.

2. The method according to claim 1, wherein the method is for treating at least one symptom of a patient suffering from Parkinson's disease selected from a motor symptom and a nonmotor symptom (NMS).

3. The method according to claim 1, wherein the method is for treating at least one symptom of a patient suffering from Parkinson's disease selected from dyskinesia, pain, and constipation.

4. The method according to claim 3, wherein the at least one symptom of a patient suffering from Parkinson's disease is dyskinesia.

5. The method according to claim 4, wherein the dyskinesia is induced by a dopaminergic.

6. The method according to claim 3, wherein the at least one symptom of a patient suffering from Parkinson's disease is pain, wherein said pain cannot be treated in said patient by further increasing the dose of a dopaminergic since this increase would concurrently result in a worsening of the side effects due to the dopaminergic.

7. The method according to claim 3, wherein the at least one symptom of a patient suffering from Parkinson's disease is constipation.

8. The method according to claim 1, wherein the opioid agonist is selected from morphine, oxycodone, hydromorphone, dihydroetorphine, etorphine, nalbuphine, propoxyphene, nicomorphine, dihydrocodeine, diamorphine, papavereturn, codeine, ethylmorphine, phenylpiperidine, methadone, dextropropoxyphene, buprenorphine, pentazocin, tilidine, tramadol, tapentadol, hydrocodone, and pharmaceutically acceptable salts thereof.

9. The method according to claim 1, wherein the opioid antagonist is selected from naltrexone, naloxone, nalmefene, nalorphine, nalbuphine, naloxonazine, methylnaltrexone, ketylcyclazocine, norbinaltorphimine, naltrindole, and pharmaceutically acceptable salts thereof.

10. The method according to claim 1, wherein the opioid antagonist has an oral bioavailability of less than about 5%.

11. The method according to claim 9, wherein the opioid antagonist is naloxone.

12. The method according to claim 1, wherein the dosage form is an oral dosage form.

13. The method according to claim 1, wherein
the opioid agonist is oxycodone or a pharmaceutically acceptable salt thereof; and
the opioid antagonist is naloxone or a pharmaceutically acceptable salt thereof.

14. The method according to claim 13, wherein
the oxycodone or a pharmaceutically acceptable salt thereof is present in an amount equivalent to 1 mg to 160 mg oxycodone HCl; and
the naloxone or a pharmaceutically acceptable salt thereof is present in an amount equivalent to 0.5 mg to 80 mg naloxone HCl.

15. The method according to claim 13, wherein the oxycodone or a pharmaceutically acceptable salt thereof and the naloxone or a pharmaceutically acceptable salt thereof are present in a 2:1 ratio by weight.

16. The method according to claim 1, wherein
the opioid agonist is hydromorphone or a pharmaceutically acceptable salt thereof; and
the opioid antagonist is naloxone or a pharmaceutically acceptable salt thereof.

17. The method according to claim 16, wherein
the hydromorphone or a pharmaceutically acceptable salt thereof is present in an amount equivalent to 1 mg to 64 mg hydromorphone HCl; and
the naloxone or a pharmaceutically acceptable salt thereof is present in an amount equivalent to 0.5 mg to 256 mg naloxone HCl.

18. The method according to claim 16, wherein the hydromorphone or a pharmaceutically acceptable salt thereof and the naloxone or a pharmaceutically acceptable salt thereof are present in a 2:1, 1:1, 1:2, or 1:3 ratio by weight.

19. The method according to claim 1, wherein the dosage form is a prolonged release dosage form.

20. The method according to claim 19, wherein the dosage form comprises a prolonged release matrix.

21. The method according to claim 19, wherein the dosage form comprises a prolonged release coating.

22. The method according to claim 20, wherein the matrix comprises a fatty alcohol and a hydrophobic polymer.

23. The method according to claim 1, wherein the dosage form is an immediate release dosage form.

24. The method according to claim 1, wherein the dosage form is selected from a tablet, a capsule, a multi-particulate, a dragée, a granulate, and a powder.

25. The method according to claim 1, wherein the method is for treating at least one symptom of a patient suffering from Parkinson's disease selected from dyskinesia, hypokinesia, rigor, and tremor.

26. The method according to claim 1, wherein the method is for treating at least one symptom of a patient suffering from Parkinson's disease selected from constipation, disturbed bowel function, urgency, nocturnia, cardiovascular symptoms, sleeping disorders, fatigue, apathy, drooling of saliva, difficulties in maintaining concentration, skin disorders, psychiatric disorders, respiratory symptoms, cough, dyspnea, and pain.
